(19) United States
(12) Patent Application Publication
(10) Pub. No.: US 2005/0222535 A1
(43) Pub. Date: Oct. 6, 2005

(54) Method and apparatus for supplying predetermined gas into body cavities of a specimen

(76) Inventors: Takefumi Uesugi, Tokyo (JP); Daisuke Sano, Tokyo (JP); Atsuhiko Kasahi, Yokohama (JP); Kenji Noda, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/093,625

(22) Filed: Mar. 30, 2005

(30) Foreign Application Priority Data
Mar. 30, 2004 (JP) 2004-100593

(51) Int. Cl. A61M 37/00; A61B 5/00

(52) U.S. Cl. 604/26; 600/560

(57) ABSTRACT

In a gas supply apparatus, a switching unit switches output of a predetermined gas to any one of first and second delivery members. A pressure regulator regulates a pressure of the predetermined gas to a first pressure suitable for the first body cavity when the output of predetermined gas is switched to the first delivery member by the switching unit. The pressure regulator regulates the pressure of the predetermined gas to a second pressure suitable for the second body cavity when the output predetermined gas is switched to the second delivery member by the switching unit.

### CROSS REFERENCE TO RELATED APPLICATION

This application is based upon the prior Japanese Patent Application 2004-100593 filed on Mar. 30, 2004 and claims the benefit of priority therefrom so that the descriptions of which are all incorporated herein by reference.

### BACKGROUND OF THE INVENTION

1. Field of the Invention

The present invention relates to a method and an apparatus for supplying predetermined gas into a body cavity of a specimen.

2. Description of the Related Art

In recent years, laparoscopic surgeries have been practiced extensively. The laparoscopic surgery is executed for treating a patient with minimally invasive capability.

Specifically, in the laparoscopic surgeries, for example, a rigid endoscope, referred to as “rigidscope”, for observation is inserted into a body cavity, such as, an abdominal cavity of a patient. A treatment tool is inserted into the abdominal cavity to be guided to a site to be treated therein while an image of the inside of the abdominal cavity, which is obtained by the rigidscope, is observed.

In such a laparoscopic surgery, an insufflator has been used for supplying carbon dioxide gas (hereinafter also referred to as CO₂) as insufflating gas into an abdominal cavity of the patient to ensure the rigidscope field and a space to manipulate the treatment tool.

Conventionally, some types of insufflators each for supplying carbon dioxide into one of body cavities, such as an abdominal cavity of the patient, have been prepared.

For example, Japanese Unexamined Patent Publication No. 2000-139830 discloses a gas supplying apparatus designed to feed a control signal to a pressure-regulating valve when gas flow volume does not reach a predetermined value. The control signal causes the pressure-regulating valve to increase the pressure of the output gas to control the amount thereof, thereby keeping an internal pressure of a living body at the predetermined value.

Moreover, Japanese Unexamined Patent Publication No. 8-256972 discloses an insufflator having a plurality of electro magnetic valves for controlling a state of gas flowing through a gas delivery channel extending from a gas supply source to an insufflation tool. Specifically, the insufflator is designed so that the plurality of electro magnetic values is integrated with a manifold valve, allowing the gas-flow state controlling section to become compact.

In the meanwhile, when diagnosing and treating a lumen, such as the stomach, the large intestine, or the like of a patient as one of the body cavities thereof, a flexible endoscope, referred to as “flexiblescope”, and a treatment tool therefor have been used. The flexiblescope has one thin and flexible end portion to be used as an access site into the lumen. The treatment tool for the flexiblescope is designed so that its forceps channel is inserted into the flexiblescope to project through an opening formed in the head of the one end portion of the flexiblescope.

When executing curative intervention, such as diagnosis and treatment of a lumen in a patient under such monitored conditions with the flexiblescope, in some cases, gas for lumens is injected into the lumen. The injection of gas aims at securing the flexiblescope field and a space to manipulate the treatment tool.

In these cases, the gas to be supplied into the lumen (organ cavity) can be transferred with a gas supply pump. As the gas for lumens, air has been generally applied, but carbon dioxide gas can be used.

Recently, as a new attempt, in the laparoscopic surgeries, the rigidscope is inserted into an abdominal cavity of a patient with the flexiblescope inserted into a lumen of the patient. This allows identification of a site to be treated in the patient based on an image of the inside of the abdominal cavity, which is obtained by the rigidscope, and that of the inside of the lumen, which is obtained by the flexiblescope.

Under such monitored conditions with both the rigidscope and flexiblescope, in some cases, for example, air as gas for lumens is injected through the flexiblescope into the lumen so that the lumen inflates.

When air is supplied into the lumen, it is difficult for the air to be absorbed into the living body. This may cause the lumen to remain inflated.

For this reason, when inserting the rigidscope into an abdominal cavity of a patient while inserting the flexiblescope into a lumen thereof, using an endoscope CO₂ regulator (hereinafter referred to as ECR) has been considered to supply carbon dioxide gas (CO₂), which is absorbed easily into the living body, into the lumen.

### SUMMARY OF THE INVENTION

The present invention has been made on the background.

According to one aspect of the present invention, there is provided a gas supply apparatus. The gas supply apparatus includes a supplier supplying predetermined gas, a first delivery member delivering the predetermined gas to a first body cavity inside a specimen, and a second delivery member delivering the predetermined gas to a second body cavity inside the specimen. The gas supply apparatus includes a pressure regulator coupled to the supplier to receive the predetermined gas supplied from the supplier. The pressure regulator regulates a pressure of the received predetermined gas to a first pressure and a second pressure. The first pressure is suitable for the first body cavity, and the second pressure is suitable for the second body cavity. The gas supply apparatus includes a switching unit coupled to the pressure regulator, and the first and second delivery members. The switching unit is configured to switch output of the predetermined gas, whose pressure is regulated by the pressure regulator, to any one of the first and second delivery members. The gas supply apparatus includes a controller electrically connected to the pressure regulator and the switching unit, and operative to control the pressure regulator and the switching unit so that the predetermined gas with the first pressure is supplied to the first delivery member and the predetermined gas with the second pressure is supplied to the second delivery member.

According to another aspect of the present invention, there is provided a gas insufflating apparatus for insufflating predetermined gas supplied from a supplier to a first body cavity of a specimen through a first delivery member and to a second body cavity of the specimen through a second delivery member. The gas insufflating apparatus comprises means for switching output of the predetermined gas to any one of the first and second delivery members, and means for regulating a pressure of the predetermined gas to a first pressure suitable for the first body cavity when output of the predetermined gas is switched to the first delivery member by the switching means. The gas insufflating apparatus includes means for regulating the pressure of the predetermined gas to a second pressure suitable for the second body cavity when the output of the predetermined gas is switched to the second delivery member by the switching means.

According to a further aspect of the present invention, there is provided an observation system. The observation system includes a gas supply apparatus. The gas supply apparatus includes a supplier supplying predetermined gas, and a first delivery member delivering the predetermined gas to a first body cavity inside a specimen. The gas supply apparatus includes a second delivery member delivering the predetermined gas to a second body cavity inside the specimen. The gas supply apparatus includes a pressure regulator coupled to the supplier to receive the predetermined gas supplied from the supplier. The pressure regulator regulates a pressure of the received predetermined gas to a first pressure and a second pressure. The first pressure is suitable for the first body cavity, the second pressure is suitable for the second body cavity. The gas supply apparatus includes a switching unit coupled to the pressure regulator, and the first and second delivery members. The switching unit is configured to switch output of the predetermined gas, whose pressure is regulated by the pressure regulator, to any one of the first and second delivery members. The gas supply apparatus includes a controller electrically connected to the pressure regulator and the switching unit, and operative to control the pressure regulator and the switching unit so that the predetermined gas with the first pressure is supplied to the first delivery member and the predetermined gas with the second pressure is supplied to the second delivery member. In addition, the observation system further includes an observation device integrated with a gas delivery channel and configured to be inserted into the second body cavity of the specimen to observe an inside of the second body cavity. The gas delivery channel serves as part of the second delivery member.

According to a still further aspect of the present invention, there is provided a method of supplying gas using a first delivery member connected into an inside of a first body cavity of a specimen and a second delivery member connected into an inside of a second body cavity of the specimen. The method includes switching output of the predetermined gas to any one of the first and second delivery members. The method includes regulating a pressure of the predetermined gas to a first pressure suitable for the first body cavity when the output of predetermined gas is switched to the first delivery member by the switching. The method includes regulating the pressure of the predetermined gas to a second pressure suitable for the second body cavity when the output predetermined gas is switched to the second delivery member by the switching.

### BRIEF DESCRIPTION OF THE DRAWINGS

Various aspects of the present invention will be more particularly described with reference to the accompanying drawings in which:

FIG. 1 is an overall structural view schematically illustrating the structure of an endoscopic surgical system equipped with a gas supply apparatus according to a first embodiment of the present invention;

FIG. 2 is a view schematically illustrating a configuration example of an operation panel illustrated in FIG. 1;

FIG. 3 is a view schematically illustrating an example of a display panel illustrated in FIG. 1;

FIG. 4 is a view schematically illustrating a configuration example of a manually operable setting section and a display section provided on a front panel of the gas supply apparatus illustrated in FIG. 1;

FIG. 5 is a block diagram illustrating a schematic structure of the gas supply apparatus illustrated in FIG. 1;

FIG. 6 is a flowchart schematically illustrating control operations of a controller illustrated in FIG. 5;

FIG. 7 is a block diagram illustrating a schematic structure of a gas supply apparatus according to a modification of the first embodiment;

FIG. 8 is a view schematically illustrating a configuration example of a manually operable setting section and a display section provided on a front panel of a gas supply apparatus according to a second embodiment of the present invention;

FIG. 9 is a block diagram illustrating a schematic structure of the gas supply apparatus according to the second embodiment of the present invention; and

FIG. 10 is a flowchart schematically illustrating control operations of a controller illustrated in FIG. 9.

### DETAILED DESCRIPTION OF EMBODIMENTS OF THE INVENTION

Various embodiments according to the present invention are described with reference to the accompanying drawings.

### First Embodiment

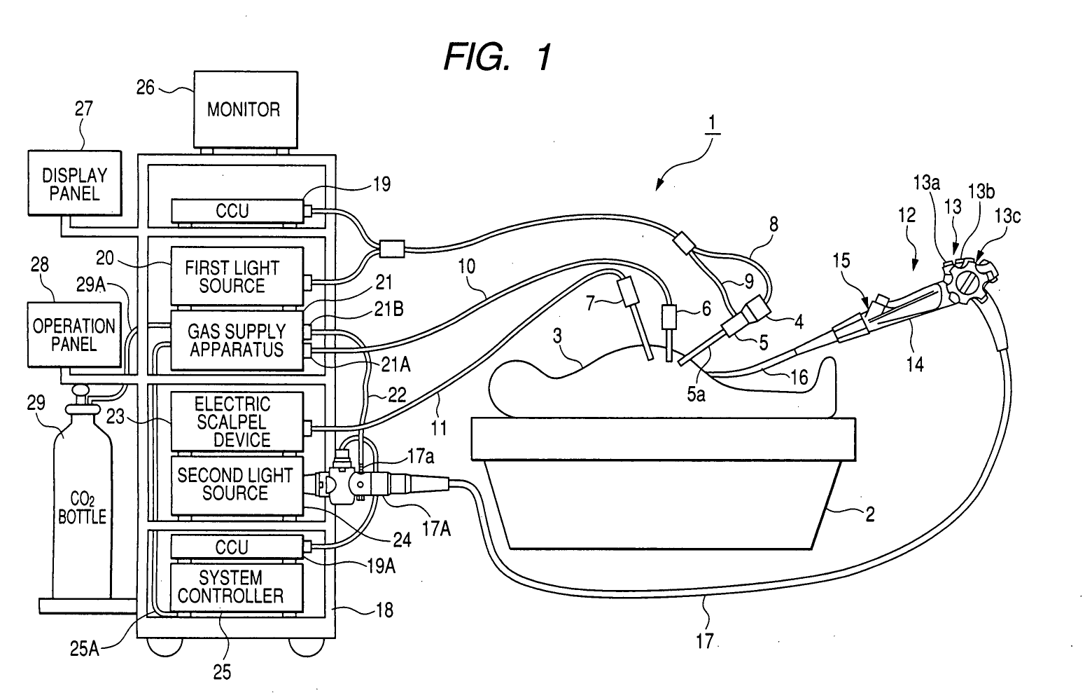

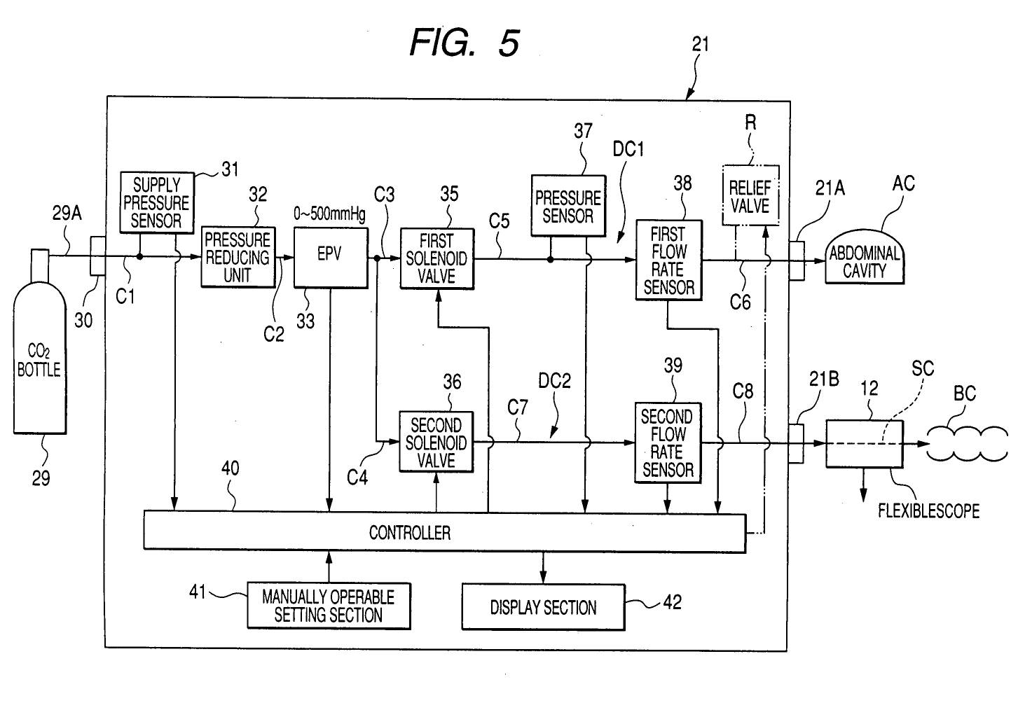

As shown in FIG. 1, an endoscopic surgical system 1 with a gas supply apparatus according to a first embodiment of the present invention has a rigid endoscope 5. The rigid endoscope is referred to as “rigidscope” hereinafter. The rigidscope 5 is integrated with a TV camera head 4 incorporating an image pickup device, such as a TV camera with a CCD (Charge Coupled Device) or the like. The rigidscope 5 has one end portion 5a designed to be inserted through a trocar (not shown) into an abdominal cavity AC (see FIG. 5) of a patient 3 as a specimen who lies on an operation table 2. The abdominal cavity AC, which means a cavity separated by the diaphragm from the thoracic cavity above and by the plane of the pelvic inlet from the pelvic cavity below, serves as a first body cavity of the patient 3 according to the first embodiment.

The endoscopic surgical system 1 is provided with an insufflation guide tube (trocar) 6. The insufflation guide tube 6 allows predetermined gas, such as carbon dioxide gas (CO₂), to be supplied into the abdominal cavity AC of the patient 3 so that the gas is insufflated therein. The insufflation of carbon dioxide gas permits the rigidscope field to be ensured in the abdominal cavity AC. The endoscopic surgical system 1 is provided with an electrical scalpel probe 7. The electrical scalpel probe 7 serves as an example of surgical tools for electrically cautery treatment on, for example, an affected site (a site to be treated) of the patient's abdominal cavity AC. The insufflation guide tube 6 and the electric scalpel probe 7 are inserted into the patient 3.

The rigidscope 5 is provided with a signal cable 8 connected to the image pickup device of the TV camera head 4. The signal cable 8 is operative to deliver a drive signal to the image pickup device and a first image signal picked up by the image pickup device.

The endoscope surgical system 1 is provided with a light guide cable 9 connected to the rigidscope 5 so that illumination light is guided through the light guide cable to the rigidscope 5. In addition, the endoscope surgical system 1 is provided with an insufflation tube (hereinafter referred to as an abdominal cavity tube) 10 whose one end is airtightly detachably connected to the abdominal insufflation guide tube 6. The abdominal cavity tube 10 is made of a material such as, for instance, silicone, Teflon®, or other similar materials. The abdominal cavity tube 10 is operative to transfer the carbon dioxide gas. Connected to the electric cautery probe 7 is a signal cable 11.

The rigidscope 5 is provided with an illumination optics (not shown) and an observation optics (not shown) installed in the one end portion thereof, respectively. The illumination optics is composed of, for example, a light guide and the like, and configured to illuminate light guided through the light guide cable 9 onto a target, such as the site to be treated, of the inside of the patient. For example, the observation optics includes relay lenses and the like.

The endoscope surgical system 1 is provided with a movable trolley 18, a first camera control unit, referred to simply as first CCU, 19, a first light source 20, a gas supply apparatus 21, and an electrical scalpel device 23. The first CCU 19, the first light source 20, the gas supply apparatus 21, and the electrical scalpel device 23 are mounted on the movable trolley 18, respectively.

The first CCU 19 is electrically connected to the image pickup device in the TV camera head 4 through the signal cable 8. The first CCU 19 is operative to execute electrical drive control of the image pickup device via the signal cable 8. The first CCU 19 is also operative to receive the first image signal to execute signal processing based on the received first image signal picked up by the image pickup device.

The first light source 20 has a function of supplying illumination light to the rigidscope 5 via the light guide cable 9.

The gas supply apparatus 21 has a function of supplying the carbon dioxide gas, as above-described predetermined gas, into both the abdominal cavity AC and a lumen BC (see FIG. 5) as a second body cavity of the patient 3. In the specification, the lumen is defined as the cavity of an organ in a specimen, such as the cavity of the stomach, the cavity of the large intestine, the cavity of a blood vessel, or the like in the specimen.

The electric scalpel device 23 is connected to the signal cable 11 and operative to apply high frequency electric power for cautery to the electric scalpel probe 7 through the signal cable 11. The electric scalpel device 23 is also referred to as “diathermy cautery device”.

In the rigidscope 5, an optical image of the target is focused on a light sensitive pixel area of the image pickup device with the illumination light incident on the target of the patient 3 via the first light source 20, the light guide fiber 9, and the illumination optics. The optical image of the target is delivered to be focused on the light sensitive pixel area of the image pickup device so that the optical image of the target is photoelectrically converted into an electric signal by the image pickup device based on the control of the first CCU 19.

The converted electric signal is transmitted, as the first image signal, to the first CCU 19 via the signal cable 8. The first CCU 19 subjects the first image signal to predetermined image processing, and after that, transmits the first image signal to a system controller described hereinafter.

The gas supply apparatus 21 is provided with a first adapter (connector) 21A airtightly connected to the other end of the abdominal cavity tube 10 such that the carbon dioxide gas supplied from the gas supply apparatus 21 is transferred through the abdominal cavity tube 10 and the abdominal insufflation tube 6 into the abdominal cavity AC.

Moreover, the endoscopic surgical system 1 is provided with a flexible endoscope, referred to as “flexiblescope”, 12 that allows endoscopic inspection of the inside of the lumen BC of the patient 3. The flexiblescope 12 has a substantially hollow-rod (tubular) shape, which is narrow in diameter and flexible. The flexiblescope 12 is internally formed with a gas delivery channel SC (see FIG. 5). The flexiblescope 12 has an image pickup device, such as a TV camera with a CCD or the like incorporated therein.

The flexiblescope 12 is provided at its one end with a manipulator 13 that allows, for example, an operator to manipulate the flexiblescope 12. The flexiblescope 12 is provided with a gripper 14 whose one end is airtightly coupled to the manipulator 13 and designed to be gripped by, for instance, an operator. The flexiblescope 12 is provided with a treatment tool insertion opening 15 formed at the gripper 14. The treatment tool insertion opening 15 allows treatment tools to be inserted therethrough.

The flexiblescope 12 is provided with an insert portion 16 that is so configured that it can be inserted into the interior of the patient 3. The insert portion 16 has one and the other ends, the one end of which is airtightly coupled to the other end of the gripper 14. The other end of the insert portion 16 is formed with a port communicated with the gas delivery channel SC so that the carbon dioxide gas transferred through the gas delivery channel SC is delivered into the lumen BC.

It should be noted that the term “operator” through the specification is not necessarily limited to a person who actually treats; the term “operator” refers to a concept that involves any nurses or other operators who assist such a treatment action.

The manipulator 13 is provided with a gas and water supply switch 13a mounted thereon. The gas and water supply switch 13a is formed with a through hole communicated with the gas delivery channel SC inside of the manipulator 13. The gas and water supply switch 13a, the gas delivery channel SC, and the insert portion 16 allow the operator to supply gas and water therethrough.

The manipulator 13 is provided with a suction switch 13b disposed thereto and a flexion knob 13c that allows the operator to flex a flexible portion (not shown) of the flexiblescope 12.

The flexiblescope 12 is provided with a universal cord 17 one end of which is airtightly connected to the other end of the manipulator 13. The universal cord 17 is integrated with a gas delivery channel (not shown) for delivery of the carbon dioxide gas. The other end of the universal cord 17 is optically connected via a connector 17A to a second light source 24 mounted on the trolley 18.

The universal cord 17 is integrated with a light guide fiber. The second light source 24 has a light source and an optical system (that are not shown). The second light source 24 is optically coupled through the connector 17A to the light guide fiber of the universal cord 17 so that illumination light supplied from the second light source 23 is transferred to the flexiblescope 12 through the connector 17A and the universal cord 17 (light guide fiber). The light guide fiber extends through the manipulator 13, the gripper 14, and the insert portion 16 to be optically coupled to an illumination optics arranged in, for example, the insert portion 16.

The connector 17A does not only serve as an illumination light transferring path for the flexiblescope 12 but also serves as a path that allows the gas supply apparatus 21 and the universal cord 17 to communicate with each other.

Specifically, the connector 17A has a carbon dioxide supply port 17a airtightly connected to the gas delivery channel inside the universal cord 17 in communication therewith.

The endoscope surgical system 1 is provided with a lumen tube 22 whose one end is connected by an airtight detachable carbon dioxide supply port 17a. The lumen tube 22 is made of a material such as, for instance, silicone, Teflon®, or other similar materials.

The gas supply apparatus 21 is provided with a second adapter (connector) 21B airtightly connected to the other end of the lumen tube 22.

With the structure of the gas supply section of the flexiblescope 12, for supplying the carbon dioxide gas through the flexiblescope 12, the operator closes the through hole of the gas and water supply switch 13a. While the through hole is closed, carbon dioxide gas is supplied from the gas supply apparatus 21 through the second adapter 21B and delivered through the lumen tube 22, the connector 17A, the universal cord 17, and the gas delivery channel SC inside the flexiblescope 12 to flow into the insert portion 16 thereof. The carbon dioxide is fed out of the port of the insert portion 16.

The endoscopic surgical system 1 includes a system controller 25 mounted on the trolley 18 and operative to perform control of the whole system 1. The endoscopic surgical system 1 includes a second CCU 19A electrically connected to the second light source 24. The second CCU 19A is operative to drive and control the image pickup device contained in the flexiblescope 12 via the universal cord 17. The second CCU 19A is also operative to subject a second image signal picked up by the image pickup device to signal processing.

Specifically, while the illumination light is illuminated on a target inside the patient, such as the lumen BC, through the light guide fiber and the illumination optics (not shown), from the second light source 24, an optical image of the target is focused on the light sensitive pixel area of the image pickup device of the flexiblescope 12.

The optical image of the target is photoelectrically converted into an electric signal by the image pickup device based on the control of the second CCU 25.

The converted electric signal is transmitted, as the second image signal, to the second CCU 25 via the signal cable 8. The second CCU 25 subjects the second image signal to predetermined image processing, and after that, transmits the second image signal to the system controller 25.

In addition, the endoscopic surgical system 1 includes a recording device, such as a VTR (Video Tape Recorder), a monitor 26 and a carbon dioxide gas cylinder (CO₂ bottle) 29 as, for example, a supplier. The VTR is operative to record the first and second image signals that are subjected to the signal processing and outputted from the first and second CCUs 19 and 19A. The monitor 26 has a function of receiving the first and second image signals outputted from the first and second CCUs 19 and 19A to display first and second images thereon based on the received first and second image signals. Specifically, the first image is an endoscopic image corresponding to the first image signal picked up by the rigidscope 5, and the second image is an endoscopic image corresponding to the second image signals picked up by the flexiblescope 12.

The CO₂ bottle 29 is connected to the gas supply apparatus 21 through a high-pressure gas tube 29A. The high-pressure gas tube 29A allows the carbon dioxide gas supplied from the CO₂ bottle 29 to be transferred into the gas supply apparatus 21 therethrough.

Furthermore, the endoscopic surgical system 1 is provided with a display panel 27 and an operation panel 28 that are mounted on the trolley 18. The operation panel 28 is, for example, a touch panel that allows the operator to set the settings of the gas supply apparatus 21, the electric cautery device 23, and the like. The display panel 27 allows display of the current settings on the gas supply apparatus 21, the electric scalpel device 23, and the like, which are set with the operation panel 28, to the operator.

The peripheral devices including the first and second CCUs 19 and 19A, the first light source 20, the electric scalpel device 23, the VTR (recording device), the display panel 27, and the operation panel 28, each of which is mounted on the trolley 18, is communicably connected to the system controller 25 through communication lines (not shown).

The operator manipulates graphical objects displayed on the operation panel 28 to set the settings of at least one of the peripheral devices connected to the system controller 25, thereby entering instructions indicative of the settings set with the panel 28 to the system controller 25. Incidentally, the endoscopic surgical system 1 may have a remote controller wirelessly communicable with the system controller 25. The remote controller allows the operator to manipulate its operations buttons to set the settings of at least one of the peripheral devices connected to the system controller 25, thereby entering instructions indicative of the settings set with the remote controller to the system controller 25.

The settings of at least one of the peripheral devices and the operating states thereof corresponding to the settings are displayed on the operation panel 28 itself and/or the display panel 27 based on the control of the system controller 25.

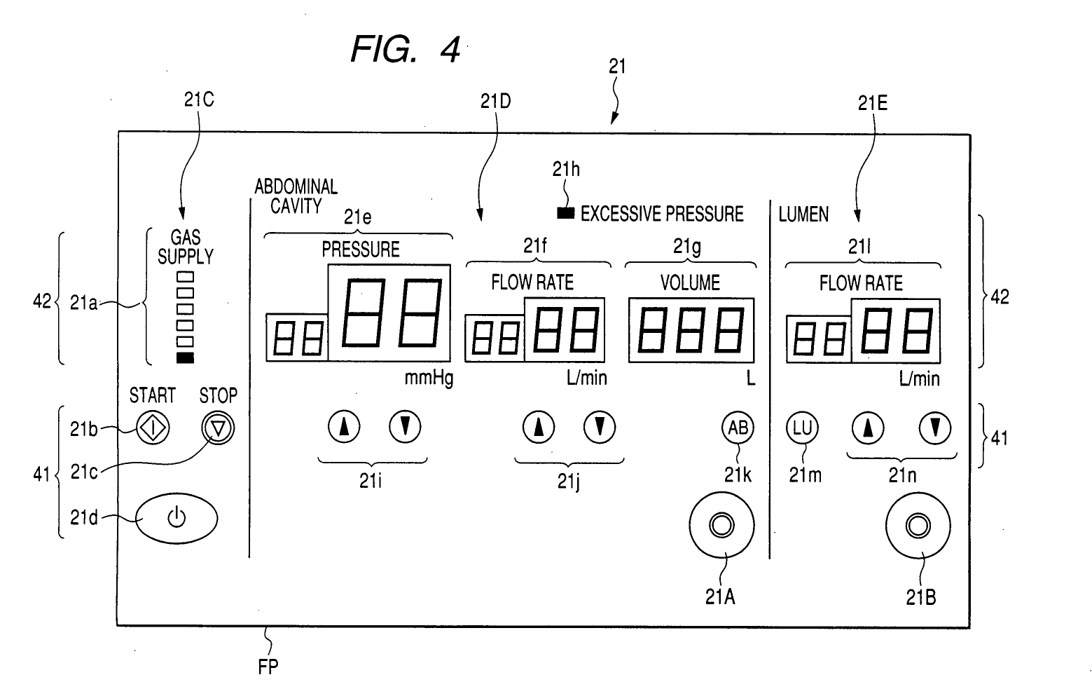

The system controller 25 has a function of receiving instructions sent from the operation panel 28 and/or a manually operable setting section 41 (see FIG. 4), which will be described hereinafter. The system controller 25 also has a function of transmitting control signals based on the received instructions to the display panel 28 and a display section 42 (see FIG. 4), described hereinafter, of the gas supply apparatus 21. The control signals cause the display panel 28 and/or the display section 42 to display the settings of at least one of the peripheral devices and the operating states thereof corresponding to the received instructions.

In addition, the system controller 25 has a function of transmitting signals indicative of information to be displayed to display panel 27 and/or the display section 42 of the gas supply apparatus 21. Furthermore, the system controller 25 has an image signal processing function. The image signal processing function sends to the monitor 26 first and second image information generated based on the first and second image signals transmitted from the first and second CCUs 19 and 19A, respectively. This allows the monitor 26 to display at least one of the first and second image information.

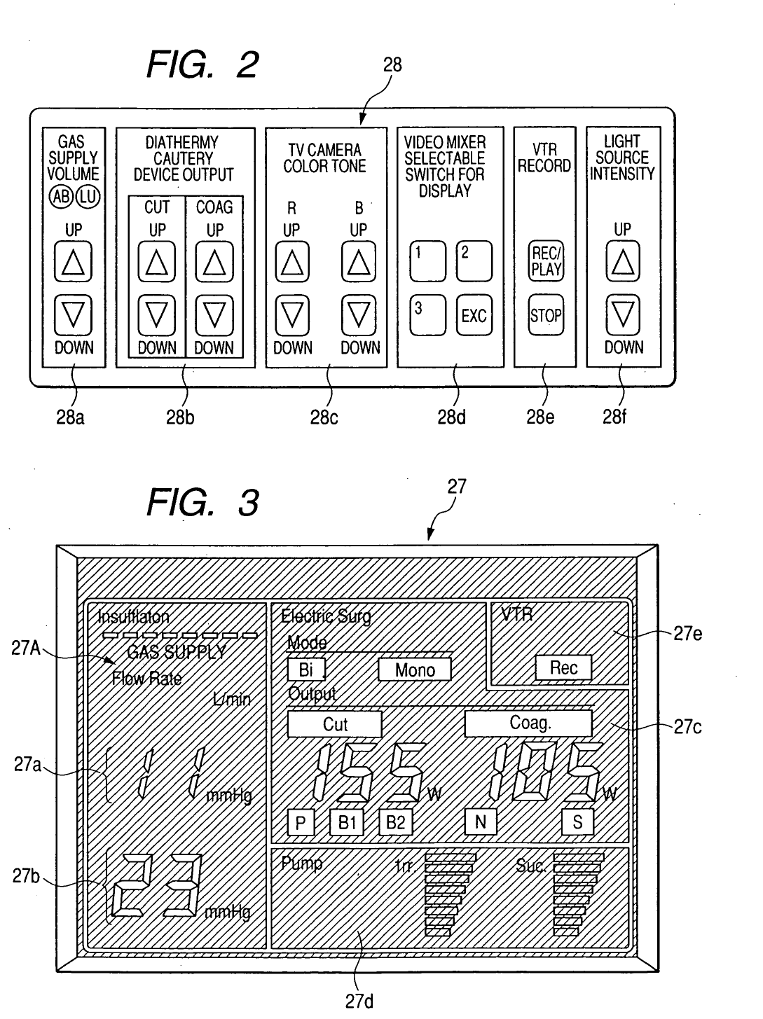

A configuration example of the operation panel 28 is illustrated in FIG. 2.

The operation panel 28 is composed of a display screen, such as a liquid crystal display, and a touch-sensitive device integrally formed on the display screen. On the display screen, manually operable sections, such as manually operable graphical buttons, are displayed. The manually operable sections allow the operator to set operating conditions (parameters) with respect to the peripheral devices to give instructions for operating them based on the set operating conditions to the system controller 25. Specifically, the operator touches at least one of the operable sections (operable buttons), with, for example, a finger so that the touch-sensitive device sets operating conditions corresponding to at least one of the touched operable sections to send to the system controller 25 instructions for operating a corresponding one of the peripheral devices based on the set operating conditions. The system controller 25 controls the corresponding one of the peripheral devices based on the instructions so that the corresponding one of the peripheral devices operates under the set operating conditions.

For example, as shown in FIG. 2, manual operation buttons 28a are graphically displayed on the display screen of the operation panel 28. The manual operation buttons 28a allow the operator to adjust the flow-rate of carbon dioxide gas supplied to the abdominal cavity AC or the lumen BC from the gas supply apparatus 21.

Manual operation buttons 28b are graphically displayed on the display screen of the operation panel 28. The manual operation buttons 28b permit the operator to adjust an output value of the electric scalpel device 23. Manual operation buttons 28c are graphically displayed on the display screen of the operation panel 28. The manual operation buttons 28c allow the operator to control color tones of the first and second CCUs 19 and 19A.

In addition, manual operation buttons 28d are graphically displayed on the display screen of the operation panel 28. The manual operation buttons 28d allow the operator to send instructions to the system controller 25 for selectively switching the first image (the endoscopic image of the rigidscope 5) and the second image (the endoscope image of the flexiblescope 12), which are displayed on the monitor 26.

Manual operation buttons 28e are graphically displayed on the display screen of the operation panel 28. The manual operation buttons 28e allow the operator to send instructions to the system controller 25 for making the VTR start recording the first image and/or second image on a video tape or for stopping the record of the first image and/or second image thereon.

Manual operation buttons 28f are graphically displayed on the display screen of the operation panel 28. The manual operation buttons 28f permit the operator to adjust light intensity of the illumination light irradiated from the first light source 20 and that of the illumination light irradiated from the second light source 24.

An example of the display panel 27 shown in FIG. 1 is illustrated in FIG. 3.

As illustrated in FIG. 3, display areas 27A (27a, 27b), 27c, 27d, and 27e are graphically represented on the display screen of the display panel 27. The display areas 27A (27a, 27b), 27c, 27d, and 27e are allocated to the gas supply apparatus 21, the electric scalpel device 23, a water pump (not shown), and the VTR, which are communicated to be controlled by the system controller 25, respectively.

The current settings of the peripheral devices and the operating states thereof are displayed on the corresponding display areas 27A, (27a, 27b), 27c, 27d and 27e, respectively. For example, the display area 27A is operative to display the settings and the operating state of the gas supply apparatus 21. Specifically, the display area 27A includes a display area 27a on which a current pressure in the lumen BC of the patient 3 is displayed, and a display area 27b on which a current pressure in the abdominal cavity AC of the patient 3 is displayed. The display area 27A also includes display areas for displaying the flow-rate (FLOW LATE) of the carbon dioxide gas supplied from the gas supply apparatus 21 and the volume (GAS SUPPLY) of the carbon dioxide gas remaining in the CO₂ bottle 29.

Next, a configuration example of the manually operable setting section 41 and the display section 42 provided on a front panel FP of the gas supply apparatus 21 is described with reference to FIG. 4. In the first embodiment, for example, the front panel FP is attached along one side of a housing of the gas supply apparatus 21.

As shown in FIG. 4, the manually operable setting section 41 and the display section 42 are graphically displayed on the front panel FP of the gas supply apparatus 21. The manually operable setting section 41 and display section 42 are divided in, for instance, three graphical setting and display sections 21C to 21E.

The setting and display section 21C serves as a supply source setting and display section that allows the operator to enter instructions related to the carbon dioxide gas supplied from the CO₂ bottle 29. In addition, the setting and display section 21C is designed to display the state of carbon dioxide gas supplied from the CO₂ bottle 29.

The setting and display section 21D serves as a setting and display section for an abdominal cavity. Specifically, the setting and display section 21D allows the operator to set parameters related to the pressure inside the abdominal cavity AC and the insufflation of carbon dioxide gas thereinto. The setting and display section 21D allows the operator to enter instructions related to the pressure inside the abdominal cavity AC and the insufflation of carbon dioxide gas thereinto. The setting and display section 21D is designed to display the state of the abdominal cavity AC depending on the carbon dioxide gas being insufflated thereinto.

The setting and display section 21E serves as a setting and display section for the lumen BC. Specifically, the setting and display section 21E allows the operator to set parameters related to the insufflation of carbon dioxide gas into the lumen BC; the setting and display section 21E is designed to display the state of the lumen BC depending on the carbon dioxide gas being insufflated thereinto.

The first adapter 21A is attached to the lower side of the setting and display section 21D of the front panel FP; the second adapter 21B is attached to the lower side of the setting and display section 21E of the front panel FP.

The setting and display section 21C is provided with a gas remaining volume indicators 21a as the display section 42. The setting and display section 21C is provided with a gas-supply start button 21b, a gas-supply stop button 21c, and a power switch 21d as the manually operable setting section 41.

The setting and display section 21D is provided with pressure displays 21e for the pressure inside the abdominal cavity AC, flow-rate displays 21f for the abdominal cavity AC, a total volume display 21g for the abdominal cavity AC, and an excessive pressure indicator 21h for the abdominal cavity AC as the display section 42.

The setting and display section 21D is provided with pressure setting buttons 21i for the pressure inside the abdominal cavity AC, flow-rate setting buttons 21j for the abdominal cavity AC, and an abdominal cavity select button 21k (see “AB” in FIG. 4) as the manually operable setting section 41.

The setting and display section 21E is provided with flow-rate displays 21l for the lumen BC as the display section 42.

The setting and display section 21E is provided with flow-rate setting buttons 21n for the lumen BC and an lumen select button 21m (see “LU” in FIG. 4) as the manually operable setting section 41.

The power switch 21d serves as a switch that permits the operator to turn power on and off to the apparatus 21. The gas-supply start button 21b serves as a button that allows the operator to send an instruction to start the supply of the carbon dioxide gas to a controller 40 described hereinafter. The gas-supply stop button 21c serves as a button that permits the operator to send an instruction to stop the supply of the carbon dioxide gas to the controller 40.

The pressure setting buttons 21i serve as buttons that allow the operator to send instructions to change the corresponding parameter (the pressure inside the abdominal cavity AC) to a pressure setting. The flow-rate setting buttons 21j serve as buttons that enable the operator to send instructions to change the corresponding parameter (the flow-rate of the carbon dioxide gas to be delivered into the abdominal cavity AC) to a flow-rate setting. The flow-rate setting buttons 21n serve as buttons that permit the operator to send instructions to change the corresponding parameter (the flow-rate of the carbon dioxide gas being delivered into the lumen BC) to a flow-rate setting.

Specifically, the pressure setting buttons 21i include an up button and a down button. Every time the operator clicks the up button, the pressure setting inside the abdominal cavity AC turns up; every time the operator clicks the down button, the pressure setting turns down. The pressure setting variably determined by the up and down buttons 21i is sent to the controller 40 every time at least one of the up and down buttons 21i is operated.

Similarly, the flow-rate setting buttons 21j include an up button and a down button. The flow-rate setting of the carbon dioxide gas to be insufflated into the abdominal cavity AC turns up every time the operator clicks the up button; the flow-rate setting turns down every time the operator clicks the down button. The flow-rate setting variably set by the up and down buttons 21j is sent to the controller 40 every time at least one of the up and down buttons 21j is operated.

Furthermore, the flow-rate setting buttons 21n include an up button and a down button. The flow-rate setting of the carbon dioxide gas to be insufflated into the lumen BC turns up every time the operator clicks the up button; the flow-rate setting turns down every time the operator clicks the down button. The flow-rate setting variably set by the up and down buttons 21n is sent to the controller 40 every time at least one of the up and down buttons 21n is operated.

The gas remaining volume indicators 21a are vertically arranged so that a top indicator that is lighting indicates the amount of carbon dioxide gas available.

The pressure displays 21e include right and left displays arranged facing toward the front panel FP. The right-side display is configured to display a pressure value (in mmHg) based on a measured value of a pressure sensor 37 described hereinafter. The left-side display is configured to display the pressure setting determined based on the operations of, for example, the pressure setting buttons 21i.

The flow-rate displays 21f include right and left displays arranged facing toward the front panel FP. The right-side display is configured to display a flow-rate (in L/min) based on a measured value of a first flow-rate sensor 38 described hereinafter. The left-side display is configured to display the flow-rate setting determined based on the operations of, for example, the flow-rate setting buttons 21j.

The total volume display 21g is configured to display a total amount of carbon dioxide gas calculated by the controller 40 based on the measured value of the first flow-rate sensor 38.

The excessive pressure indicator 21h consists of, for example, red LED (light emitting device). The excessive pressure indicator 21h is configured to turn on or flash on and off based on a control signal sent from the controller 40 at anytime the pressure measured by the pressure sensor 37 exceeds a threshold value of the pressure inside the abdominal cavity AC by a predetermined pressure. The turning-on or the flashing of the excessive pressure indicator 21h allows the operator to visually recognize that the pressure inside the abdominal cavity AC exceeds the threshold value by the predetermined pressure or more.

When the operator turns on the abdominal cavity select button 21k, the button 21k is configured to send to the controller 40 an instruction to make it execute operations for supplying the carbon dioxide gas into the abdominal cavity AC. In other words, when the operator turns on the abdominal cavity select button 21k, the button 21k is configured to send to the controller 40 an instruction to change the operation mode thereof to an abdominal cavity insufflation mode.

The flow-rate displays 21l include right and left displays arranged facing toward the front panel FP. The right-side display is configured to display a flow-rate (in L/min) based on a measured value of a second flow-rate sensor 39 described hereinafter. The left-side display is configured to display the flow-rate setting determined based on the operations of, for example, the flow-rate setting buttons 21n.

When the operator turns on the lumen select button 21m, the button 21m is configured to send to the controller 40 an instruction to make it execute operations for supplying the carbon dioxide gas into the lumen BC. In other words, when the operator turns on the lumen select button 21m, the button 21m is configured to send to the controller 40 an instruction to change the operation mode thereof to an lumen insufflation mode.

Incidentally, an excessive pressure indicator that is the same as the excessive pressure sensor 21h may be provided on the setting and display section 21E.

The structures of the manually operable setting section 41 and the display section 41 in the front panel FP allow the operator to easily give instructions to the controller 40 and to easily visually recognize the parameters related to the abdominal cavity AC and the lumen BC.

Next, a structure of the gas supply apparatus 21 will be described hereinafter with reference to FIG. 5.

As shown in FIG. 5, the gas supply apparatus 21 includes a high pressure adapter 30, a first delivery channel C1, a supply pressure sensor 31, and a pressure reducing unit 32 serving as, for example, a pressure regulator. The gas supply apparatus 21 includes a second delivery channel C2, an electropneumatic proportional valve (EPV) 33 as an example of pressure regulating valves, serving as the pressure regulator, a third delivery channel C3, and a fourth delivery channel C4.

In addition, the gas supply apparatus 21 includes first and second electromagnetic valves (solenoid valves) 35 and 36 as examples of open/close valves. The first and second solenoid valves 35 and 36 serve as a switching unit.

The gas supply apparatus 21 includes a fifth delivery channel C5, a sixth delivery channel C6, the pressure sensor 37, and the first and second flow-rate sensors 38 and 39. Moreover, the gas supply apparatus 21 includes a seventh delivery channel C7, an eighth delivery channel C8, the controller 40, the manually operable setting section 41, the display section 42, the first and second adapters 21A and 21B.

The CO₂ bottle 29 has a discharge port (cock) to which one end of the high-pressure gas tube 29A is joined. The other end of the high-pressure gas tube 29A is joined to the high-pressure adapter 30. The high-pressure adapter 30 is joined to an inlet of the pressure reducing unit 32 via the first delivery channel C1. The supply pressure sensor 31 is attached to the first delivery channel C1. An outlet of the pressure reducing unit 32 is coupled to an inlet of the electropneumatic proportional valve 33 via the second delivery channel C2. An outlet of the electropneumatic proportional valve 33 is coupled to both the third delivery channel C3 for the abdominal cavity AC and the fourth delivery channel C4 for the lumen BC.

The third delivery channel C3 is coupled to an inlet of the first solenoid valve 35 whose outlet is coupled to the fifth delivery channel C5 to which the pressure sensor 37 is attached. The fifth delivery channel C5 is coupled to an inlet of the first flow rate sensor 38 whose outlet is coupled to the first adapter 21A via the sixth delivery channel C6.

In the meanwhile, the fourth delivery channel C4 for the lumen BC is coupled to an inlet of the second solenoid valve 36 whose outlet is connected to the seventh delivery channel C7.

The seventh delivery channel C7 is coupled to an inlet of the second flow-rate sensor 39 whose outlet is coupled to the second adapter 21B via the eighth delivery channel C8.

When the cock of the CO₂ bottle 29 is opened, carbon dioxide stored therein in a liquid form is vaporized to form the carbon dioxide gas. The carbon dioxide gas is delivered to the pressure reducing unit 32 through the high-pressure gas tube 29A, the high pressure adapter 30, and the first delivery channel C1 of the gas supply apparatus 21. The carbon dioxide gas is reduced in pressure by the pressure reducing unit 32 to have a predetermined pressure, and thereafter, guided via the second delivery channel C2 to the electropneumatic proportional valve 33. The electropneumatic proportional valve 33 regulates the pressure of the carbon dioxide gas to a pressure within a range suitable for supply into the inside of the abdominal cavity AC or that of the lumen BC.

More particularly, the electropneumatic proportional valve 33 is provided with a solenoid composed of, for example, a magnet coil (solenoid coil) and a compass needle, which are not shown. The electropneumatic proportional valve 33 is provided with a thin film for pressure control, and a pressure reducing spring. The solenoid is electrically connected to the controller 40. The electropneumatic proportional valve 33 is configured such that the solenoid controls force applied on the thin film by the pressure reducing spring depending on a control signal applied from the controller 40, thereby regulating the pressure of the carbon dioxide gas.

Specifically, the electropneumatic proportional valve 33 is designed to change its opening in proportional to a voltage or a current as the control signal applied from the controller 40 so as to regulate the pressure and the flow-rate of the carbon dioxide gas flowing therethrough within the corresponding appropriate ranges, respectively.

For example, the electropneumatic proportional valve 33 allows the pressure of the carbon dioxide gas to be regulated within a range from 0 to 500 mmHg based on the control signal applied from the controller 40.

For example, the range of the pressure of the carbon dioxide gas to be insufflated into the abdominal cavity AC is preferably 0 to 80 mmHg or thereabout; the range of the flow-rate thereof to be insufflated thereinto is preferably 0.1 to 35 L/min or thereabout. Moreover, for example, the range of the pressure of the carbon dioxide gas to be insufflated into the lumen BC is preferably 100 to 500 mmHg or thereabout; the range of the flow-rate thereof to be insufflated thereinto is preferably 1 to 3 L/min or thereabout.

The carbon dioxide gas whose pressure is regulated by the electropneumatic proportional valve 33 is divided into two parts, and they are introduced into the third and fourth delivery channels C3 and C4, respectively. The third and fourth delivery channels C3 and C4 constitute bifurcating cannels, respectively. The divided parts of the carbon dioxide gas are introduced into two supply paths constituting a first CO₂ supply path DC1 directing the carbon dioxide gas into the abdominal cavity AC and a second CO₂ supply path DC2 directing it into the lumen BC, respectively.

Specifically, the downstream side of the electropneumatic proportional valve 33 is separated into the first CO₂ supply path DC1 and the second CO₂ supply path DC2 through the third and fourth delivery channels C3 and C4.

The first CO₂ supply path DC1 for the abdominal cavity AC includes the first solenoid valve 35, the fifth delivery channel C5, the first flow rate sensor 38, the sixth delivery channel C6, the first adapter 21A, the abdominal cavity tube 10, and a delivery channel (gas delivery member) provided in the insufflation guide tube 6. This configuration of the first CO₂ supply path DC1 allows the carbon dioxide gas to be introduced into the abdominal cavity AC therethrough.

The second CO₂ supply path DC2 for the lumen BC includes the second solenoid valve 36, the seventh delivery channel C7, the second flow rate sensor 39, the eighth delivery channel C8, the second adapter 21B, the lumen tube 22, the connector 17A, the universal cord 17 and the gas delivery channel SC of the flexiblescope 12. This configuration of the second CO₂ supply path DC2 permits the carbon dioxide gas to be introduced into the lumen BC therethrough.

Incidentally, in the first embodiment, a first delivery member of the present invention corresponds to at least the fifth and sixth delivery channels C5 and C6 in the first CO₂ supply path DC1. Specifically, the concept of the first delivery member of the present invention can expand to cover the whole of the first CO₂ supply path DC1 depending on aspects of the gas supply apparatus 21.

Likewise, in the first embodiment, a second delivery member of the present invention corresponds to at least the seventh and eighth delivery channels C7 and C8 in the second CO₂ supply path DC2. Specifically, the concept of the second delivery member of the present invention can expand to cover the whole of the second CO₂ supply path DC2 depending on aspects of the gas supply apparatus 21.

More specifically, the carbon dioxide gas with pressure regulated by the electropneumatic proportional valve 33, which flows through the third delivery channel C3, is delivered to the first solenoid valve 35. While the first solenoid valve 35 is kept opened, the carbon dioxide gas is guided to the abdominal cavity tube 10 via the first solenoid valve 35, the fifth delivery channel C5, the first flow rate sensor 38, the sixth delivery channel C6 and the first adapter 21A.

The carbon dioxide gas with pressure regulated by the electropneumatic proportional valve 33, which flows through the fourth delivery channel C4, is delivered to the second electromagnet valve 36. While the second electromagnetic valve 36 is kept opened, the carbon dioxide gas is guided to the lumen tube 22 via the second electromagnetic valve 36, the seventh delivery channel C7, the second flow rate sensor 39, the eighth delivery channel C8 and the second adapter 21B.

Because the through hole is formed at the gas and water supply switch 13a of the flexiblescope 12 to which the lumen tube 22 is connected set forth above, closing the through hole by the operator allows the carbon dioxide gas to be supplied into the lumen BC via the lumen tube 22 and the flexiblescope 12.

The supply pressure sensor 31 is electrically connected to the controller 40. The supply pressure sensor 31 has a function of detecting the pressure of the carbon dioxide gas flowing from the CO₂ bottle 29 to the first delivery channel C1 to send the detected result (detected pressure value) to the controller 40.

The pressure sensor 37 is electrically connected to the controller 40. The pressure sensor 37 has a function of measuring a pressure in the fifth delivery channel C5, in other words, a pressure inside the abdominal cavity AC when the first electromagnetic valve 35 is closed, thereby sending the measured result to the controller 40.

Each of the first and second solenoid valves 35 and 36 is electrically connected to the controller 40 and configured to open and close based on control signals sent from the controller 40. The opening and closing of the first solenoid valve 35 allow the fifth delivery channel C5 to open and close, respectively. Similarly, the opening and closing of the second solenoid valve 36 permit the seventh delivery channel C7 to open and close, respectively.

The first and second flow rate sensors 38 and 39 are electrically connected to the controller 40. The first flow rate sensor 38 has a function of detecting the flow rate of the carbon dioxide gas flowing through the first solenoid valve 35 and the fifth delivery channel C5. Similarly, the second flow rate sensor 39 is operative to detect the flow rate of the carbon dioxide gas flowing through the second solenoid valve 36 and the seventh delivery channel C7. Each of the first and second flow rate sensors 38 and 39 is configured to send the detected result to the controller 40.

The controller 40 is operative to receive the measured values outputted from the supply pressure sensor 31, the pressure sensor 37, the first and second flow rate sensors 38 and 39. The controller 40 is programmed to execute opening control (pressure control) of the electropneumatic proportional valve 33, opening and closing controls of each of the first and second solenoid valves 35 and 36, and display control of the display section 42 based on the received measured values.

In addition, the manually operable setting section 41 is electrically connected to the controller 40. The controller 40 is also programmed to execute opening control (pressure control) of the electropneumatic proportional valve 33, opening and closing controls of each of the first and second solenoid valves 35 and 36, and display control of the display section 42 based on the instructions sent from the manually operable setting section 41.

Incidentally, as shown in FIG. 5, a relief valve (opening and closing valve) R can be provided at the midstream of the sixth delivery channel C6 between the first flow rate sensor 38 and the first adapter 21A, which is illustrated as double dot lines. In this modification, the relief valve R is electrically connected to the controller 40. The relief valve R is operative to remain in a closed state, and to open based on a control signal sent from the controller 40 when the measured value of the pressure sensor 37 exceeds the predetermined threshold value. The opening of the relief valve R causes carbon dioxide gas in the abdominal cavity AC to be released, thereby reducing a pressure inside the abdominal cavity AC. Like the abdominal cavity side, a relief valve can be provided at the midstream of the eighth delivery channel C8 between the second flow rate sensor 39 and the second adapter 21B.

Incidentally, in the first embodiment, the channels and the like constituting the first CO₂ supply path DC1 provide airtight junction therebetween, and the channels and the like constituting the second CO₂ supply path DC2 provide airtight junction therebetween.

Next, operations of the gas supply apparatus 21 of the first embodiment will be described hereinafter.

The gas supply apparatus 21 of the first embodiment is used for the endoscopic surgical system 1 as illustrated in FIG. 1.

For example, when carrying out laparoscopic surgery employing the endoscopic surgical system 1, the operator inserts the rigidscope 5 into the inside of the abdominal cavity AC with the flexiblescope 12 being inserted into the lumen BC, such as a large intestine present in the abdominal cavity AC. The operator specifies and treats at least one site to be treated in the abdominal cavity AC and/or the lumen BC based on the first and second images picked up by the rigidscope 5 and the flexiblescope 12, respectively.

Specifically, the operator operates, for example, the abdominal cavity select button 21k and the gas-supply start button 21b so that the instructions corresponding to the buttons 21k and 21b are sent to the controller 40 via the manually operable setting section 41. The controller 40 executes abdominal-cavity pressure control based on the instructions by controlling the opening of the electropneumatic proportional valve 33 to regulate a pressure inside the abdominal cavity AC. The abdominal cavity pressure control allows the carbon dioxide gas to be supplied thereinto via the first CO₂ supply path DC1 with its pressure regulated to be suitable for the insufflation inside the abdominal cavity AC.

The abdominal-cavity pressure control to be executed by the controller 40 refers to, for example, a feedback control described hereinafter.

That is, the controller 40 controls the electropneumatic proportional valve 33 to adjust the opening thereof depending on the pressure value indicative of the measured result sent from the pressure sensor 37. The adjustment of the opening of the valve 33 allows the pressure value indicative of the measured result sent from the pressure sensor 37 to be maintained to the pressure setting determined by the operation of the pressure setting buttons 21i by the operator.

More particularly, the controller 40 receives the value of the abdominal-cavity pressure measured by the pressure sensor 37 and supplied therefrom when the electropneumatic proportional valve 33 is closed with no carbon dioxide gas being supplied. Next, the controller 40 controls the electropneumatic proportional valve 33 to open it. Subsequently, the controller 40 has regulated the opening of the valve 33 based on the received value of the abdominal-cavity pressure to supply the carbon dioxide gas through the electropneumatic proportional valve 33 and the first CO₂ supply path DC1 for a predetermined period of time.

Next, after the predetermined period of time has elapsed, the controller 40 controls the electropneumatic proportional valve 33 to close it again, and retrieves the value of the abdominal-cavity pressure measured by the pressure sensor 37. The controller 40 controls the opening of the electropneumatic proportional valve 33 depending on the retrieved value of the abdominal-cavity pressure to have continued supply of the carbon dioxide gas for a predetermined period of time. After the predetermined period of time has elapsed, the controller 40 controls the electropneumatic proportional valve 33 to close it.

That is, the controller 40 has repeatedly executed the feedback control set forth above to reach the pressure inside the abdominal cavity AC to the pressure setting determined by the operation of the pressure setting buttons 21i by the operator and to maintain it thereto.

In addition, the controller 40 also executes abdominal-cavity flow rate control.

Specifically, the controller 40 controls the opening of the valve 33 based on the value of the flow-rate of the carbon dioxide gas flowing through the fifth delivery channel C5 in addition to the retrieved value of the abdominal-cavity pressure. The adjustment of the opening of the valve 33 allows the flow-rate indicative of the measured result sent from the first flow-rate sensor 38 to be maintained within the predetermined range of, for example, 0.1 to 35 L/min or thereabout.

On the other hand, when the operator operates, for example, the lumen select button 21m and the gas-supply start button 21b so that the instructions corresponding to the buttons 21m and 21b are sent to the controller 40 via the manually operable setting section 41.

The controller 40 executes lumen flow-rate control based on the instructions by controlling the opening of the electropneumatic proportional valve 33 to regulate a flow-rate of the carbon dioxide gas flowing through the seventh delivery channel C7. The lumen flow-rate control allows the carbon dioxide gas to be supplied thereinto via the second CO₂ supply path DC2 with its flow-rate regulated to be suitable for the insufflation inside the lumen BC.

Specifically, while the carbon dioxide gas is supplied into the lumen BC through the valve 33 and the second CO₂ supply path DC2, the controller 40 controls the opening of the valve 33 based on the value of the flow-rate of the carbon dioxide gas flowing through the seventh delivery channel C7. The adjustment of the opening of the valve 33 allows the flow-rate indicative of the measured result sent from the second flow-rate sensor 38 to be maintained within the predetermined range of, for example, 1 to 3 L/min or thereabout.

In addition, before laparoscopic surgery, open of the cock of the CO₂ bottle 29 causes the carbon dioxide gas to flow out of the bottle 29 through the high-pressure gas tube 29A. The carbon dioxide gas flows into the gas supply apparatus 21 to be introduced through the first delivery channel C1 to the pressure reducing unit 32.

The carbon dioxide gas is reduced in pressure by the pressure reducing unit 32 to have the predetermined pressure, thereby being guided via the second delivery channel C2 to the inlet of the electropneumatic proportional valve 33.

Under a state before executing laparoscopic surgery, the electropneumatic proportional valve 33 remains closed, which causes the carbon dioxide gas not to flow any delivery channels downstream of the electropneumatic proportional valve 33.

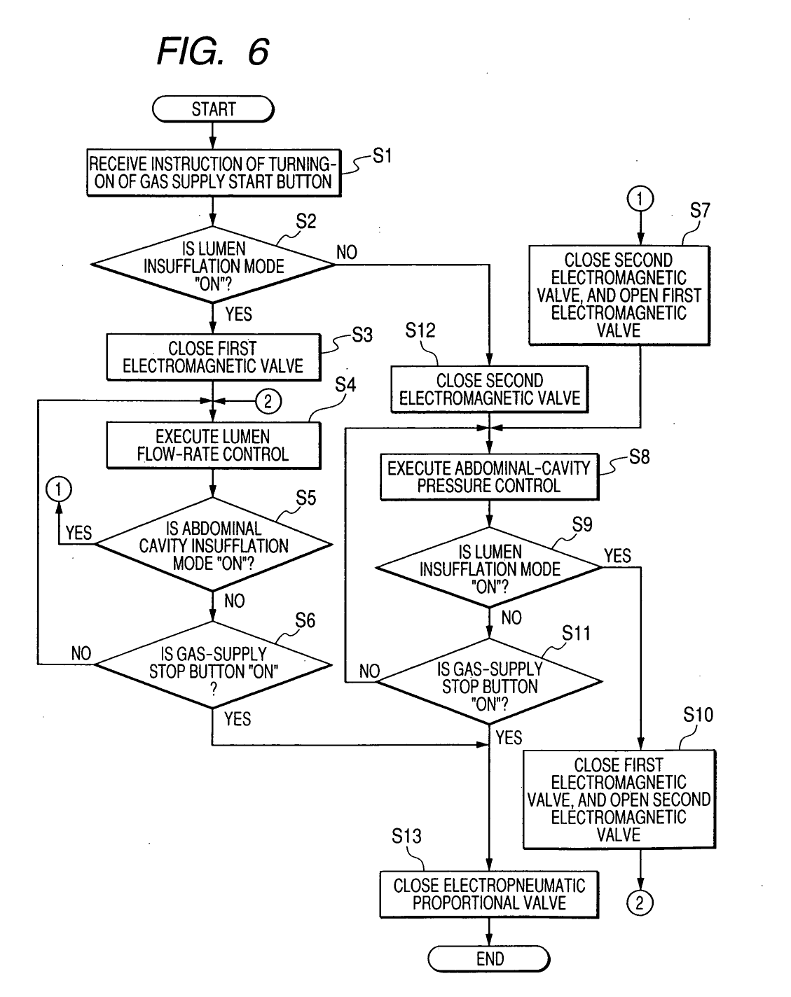

Next, specific control operations of the controller 40 of the gas supply apparatus 21 will be described hereinafter with reference to a flowchart shown in FIG. 6.

When actually starting laparoscopic surgery and picking up an image inside the lumen BC with the flexiblescope 12, the operator turns on the gas-supply start button 21b and the lumen select button 21m on the front panel FP of the gas supply apparatus 21. The manually operable setting section 41 provides the instructions corresponding to the turning-on operations of the buttons 21b and 21m to the controller 40.

The controller 40 receives the instructions corresponding to the turning-on operations of the button 21m and 21b provided from the manually operable setting section 41 proceeds to step S2 (FIG. 6; step S1). Incidentally, it is assumed that the first and second electromagnetic valves 35 and 36 are kept opened under their initial conditions whereas the electropneumatic proportional valve 33 is kept closed.

Next, the controller 40 determines whether the lumen select button 21m is turned on (step S2).

When the controller 40 determines that the lumen select button 21m is turned on, in other words, the determination in step S2 is YES, the controller 40 enters the lumen insufflation mode. In the lumen insufflation mode, the controller 40 sends the control signal to the first electromagnetic valve 35 to close it (step S3).

Subsequently, the controller 40 sends the control signal to the electropneumatic proportional valve 33 to open it, thereby sending the control signal based on the lumen insufflation mode to execute the lumen flow-rate control set forth above (step S4).

As described above, during the lumen flow-rate control, the controller 40 controls the electropneumatic proportional valve 33 to adjust the opening thereof. The adjustment of the opening of the electropneumatic proportional valve 33 allows the pressure and the flow-rate of the carbon dioxide gas to be regulated within the corresponding predetermined ranges suitable for the insufflation of the lumen BC, respectively. The carbon dioxide gas with its pressure and flow-rate being regulated each is introduced through the third and fourth delivery channels C3 and C4 to the first and second CO₂ supply paths DC1 and DC2 constituting bifurcating cannels, respectively.

Because the first electromagnetic valve 35 is closed, no carbon dioxide gas is supplied to the first CO₂ supply path DC1 for the abdominal cavity AC so that the carbon dioxide gas is introduced to the second CO₂ supply path DC2 for the lumen BC. The carbon dioxide gas is delivered toward the lumen BC through the second electromagnet valve 36, the second flow rate sensor 39, the second adapter 21B, the lumen tube 22, the connector 17A, the universal cord 17 and the gas delivery channel SC provided in the flexiblescope 12.

While the carbon dioxide gas is delivered through the second CO₂ supply path DC2 toward the lumen BC, close of the through hole of the manipulator 13 by the operator allows the carbon dioxide gas to be supplied into the lumen BC.

While the carbon dioxide gas is supplied into the lumen BC through the second CO₂ supply channel DC2, the second flow-rate sensor 39 measures the flow rate of the carbon dioxide gas flowing across the second electromagnetic valve 36 through the seventh delivery channel C7. The second flow-rate sensor 39 sends the measured result to the controller 40. The controller 40 receives the measured result. The controller 40 controls the opening of the electropneumatic proportional valve 33 based on the measured result. The control of the opening of the valve 33 causes the flow-rate of the carbon dioxide gas into the lumen BC to be regulated within the predetermined range of, for example, approximately 1 to 3 L/min set forth above, thereby controlling the flow rate in the lumen BC and the pressure inside it.

During the lumen flow-rate feedback control, the controller 40 determines whether the abdominal cavity select button 21k is turned on (step S5). When it is determined that the abdominal cavity select button 21k is not turned on, in other words, the determination in step S5 is NO, the controller 40 determines whether the gas-supply stop button 21c is turned on (step S6).

When the operator decides to continuously execute the lumen flow-rate control, because any switches 21k and 21b are not manipulated, each of the determinations in steps S5 and S6 is NO so that the controller 40 continues executing the lumen flow-rate control.

On the other hand, for example, when the operator wants to shift the operation mode from the lumen insufflation mode to the abdominal-cavity insufflation mode, the operator turns on the abdominal cavity select button 21k on the front panel FP of the gas supply apparatus 21. The manually operable setting section 41 provides the instruction corresponding to the turning-on operation of the button 21k to the controller 40.

In this case, the controller 40 receives the instruction corresponding to the turning-on of the button 21k provided from the manually operable setting section 41 so as to determine that the abdominal cavity select button 21k is turned on (the determination in step S5 is YES), automatically shifting into the abdominal-cavity insufflation mode.

Specifically, the controller 40 sends the control signal to the second electromagnetic valve 36 to close it, and sends the control signal to the first electromagnetic valve 35 to open it (step S7). Subsequently, the controller 40 sends the control signal based on the abdominal-cavity insufflation mode to the electropneumatic proportional valve 33 to execute the abdominal-cavity pressure control set forth above (step S8).

As described above, during the abdominal-cavity pressure control, the controller 40 controls the electropneumatic proportional valve 33 to adjust the opening thereof. The adjustment of the opening of the electropneumatic proportional valve 33 allows the pressure and the flow-rate of the carbon dioxide gas to be regulated within the corresponding predetermined ranges suitable for the insufflation of the abdominal cavity AC, respectively. The carbon dioxide gas with its pressure and flow-rate being regulated each is introduced through the third and fourth delivery channels C3 and C4 to the first and second CO₂ supply paths DC1 and DC2 constituting bifurcating cannels, respectively.

Because the second electromagnetic valve 36 is closed, no carbon dioxide gas is supplied to the second CO₂ supply path DC2 for the lumen BC so that the carbon dioxide gas is introduced to the first CO₂ supply path DC1 for the abdominal cavity AC. The carbon dioxide gas is delivered into the abdominal cavity AC through the first electromagnet valve 35, the first flow rate sensor 38, the first adapter 21A, the abdominal cavity tube 10 and the delivery channel provided in the abdominal-cavity guide tube 6.

While the carbon dioxide gas is delivered through the first CO₂ supply path DC1 into the abdominal cavity AC, the first flow rate sensor 38 measures the flow rate of the carbon dioxide gas flowing across the first electromagnetic valve 35 through the fifth delivery channel C5. The first flow-rate sensor 38 sends the measured result to the controller 40.

Similarly, while the carbon dioxide gas is delivered through the first CO₂ supply path DC1 into the abdominal cavity AC, the pressure sensor 37 measures the pressure inside the abdominal cavity AC when the first electromagnetic valve 35 is closed. The pressure sensor 37 sends the measured result to the controller 40.

The controller 40 receives the measured results provided from the sensors 37 and 38. The controller 40 controls the opening of the electropneumatic proportional valve 33 based on the measured results to regulate the pressure of the carbon dioxide gas and the flow-rate thereof supplied into the abdominal cavity AC within the corresponding predetermined range of approximately 0 to 80 mmHg and that of approximately 0.1 to 35 L/min, respectively.

During the abdominal-cavity pressure feedback control, the controller 40 determines whether the lumen select button 21m is turned on (step S9).

When the operator wants to shift the operation mode from the abdominal-cavity insufflation mode to the lumen insufflation mode, the operator turns on the lumen select button 21m on the front panel FP of the gas supply apparatus 21 again. The manually operable setting section 41 provides the instruction corresponding to the turning-on operation of the button 21m to the controller 40 again.

The controller 40 receives the instruction corresponding to the turning-on of the button 21m provided from the manually operable setting section 41 so as to determine that the lumen select button 21m is turned on (the determination in step S9 is YES), automatically shifting into the lumen insufflation mode.

Specifically, the controller 40 sends the control signal to the first electromagnetic valve 35 to close it, and sends the control signal to the second electromagnetic valve 35 to open it (step S10). After the operation in step S10, the controller 40 executes the lumen flow-rate control shown in step S4 and thereafter.

On the other hand, when the lumen select button 21m is not turned on, it is determined that the determination in step S9 is NO, the controller 40 determines whether the gas-supply stop button 21c is turned on (step S11).

When determining to continuously perform the abdominal-cavity pressure control, the operator does not turn on any buttons 21m and 21c (the determination in step S11 is NO), the controller 40 continues to execute the abdominal-cavity pressure control shown in step S8.

On the other hand, when determining that no lumen select button 21m is turned on, in other words, the button 21m is in off state, the determination in step S2 is NO, so that the controller 40 sends the control signal to the second electromagnetic valve 36 to close it. Subsequently, the controller 40 sends the control signal to the electropneumatic proportional valve 33 to open it (step S12), thereby executing the body-cavity pressure control shown in step S8.

Thus, the body-cavity pressure control in the abdominal cavity insufflation mode and the lumen flow-rate control in the lumen insufflation mode are carried out by the controller 40. During the body-cavity pressure control and the lumen flow-rate control, when the operator determines that it eliminates the need for supplying the carbon dioxide gas into both the abdominal cavity AC and the lumen BC, the operator turns on the gas-supply stop button 21c on the front panel FP. For example, when observations and treatments for at least one site to be treated are completed, the operator turns on the gas-supply stop button 21c on the front panel FP. The manually operable setting section 41 provides the instruction corresponding to the turning-on operation of the button 21c to the controller 40.

Accordingly, each of the determinations of the controller 40 in steps S6 and S11 is YES, so that the controller 40 sends the control signal to the electropneumatic proportional valve 33 to close it (step S13), thereby terminating the operations shown in FIG. 6.

As described above, in the first embodiment, the controller 40 executes the abdominal-cavity pressure control in the abdominal-cavity insufflation mode and the lumen flow-rate control in the lumen insufflation mode depending on the operator's needs. This allows the carbon dioxide gas whose pressure and flow-rate are regulated suitably to the abdominal cavity AC to be supplied thereinto, as well as the carbon dioxide gas whose pressure and flow-rate are regulated suitably to the lumen BC to be supplied thereinto.

The operator, therefore, can easily rapidly specify at least one site to be treated in the patient 3 while observing the first image corresponding to the inside of the abdominal cavity AC being sufficiently insufflated and the second image corresponding to the inside of the lumen BC being sufficiently insufflated. It is possible for the operator to treat the specified at least one site in the patient using, for example, the electrical scalpel probe 7.

It should be concluded, from what has been described above, the gas supply apparatus 21 serves both as an insufflator supplying the carbon dioxide gas into the abdominal cavity AC and as an ECR (endoscope CO₂ regulator) supplying it into the lumen BC. This allows the carbon dioxide gas to be supplied into the abdominal cavity AC with its pressure and flow-rate regulated suitably thereto, as well as the carbon dioxide gas to be supplied into the lumen BC with its pressure and flow-rate regulated suitably thereto.

Accordingly, as compared with a conventional system configured to control the pressure inside an abdominal cavity in a patient and that inside an lumen therein using an insufflator and an ECR, the first embodiment of the present invention makes the structure of the gas supply apparatus 21 compact, and allows the cost thereof to decrease. In addition, as compared with the conventional system, the first embodiment of the present invention enables preparations for setting up the gas supply apparatus 21 to be reduced. Moreover, in contrast the conventional system, the first embodiment of the present invention makes it possible to reduce a space where the apparatus 21 is occupied in an operating room, in other words, to increase empty space in the operating room.

Incidentally, in the first embodiment, at least one of the operations corresponding to steps S3, S7 and S12 of the controller 40, the first electromagnetic valve 35, and the second electromagnetic valve 36 correspond to an example of switching means according to the present invention. At least one of the operations corresponding to the steps S4 and S8, the pressure reducing unit 32, and the electropneumatic proportional valve 33 correspond to an example of means for selective regulation according to the present invention.

Moreover, in the first embodiment, the second CO₂ supply path DC2 includes the second electromagnetic valve 36, the seventh delivery channel C7, the second flow rate sensor 39, the eighth delivery channel C8, and the second adapter 21B. In addition, the second CO₂ supply path DC2 includes the body cavity tube 22, the connector 17A, the universal cord 17, and the gas delivery channel SC of the flexiblescope 12. The present invention is however limited to the structure.

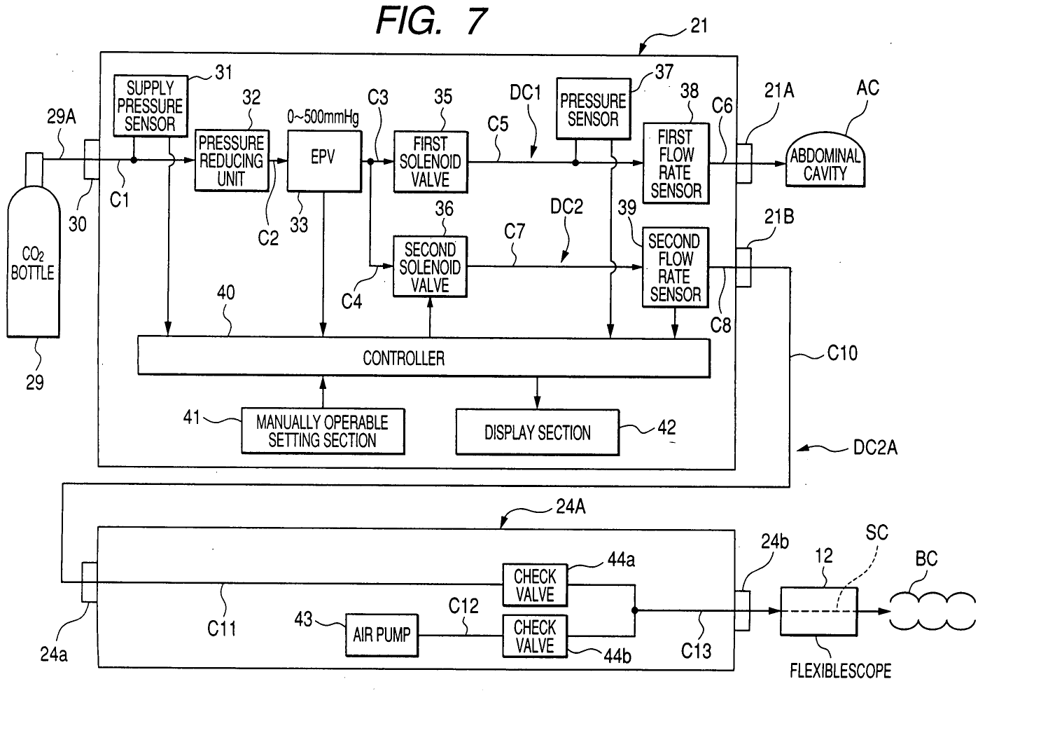

For example, as shown in FIG. 7, a conventional light source integrated with an air pump is transformed into a second light source 24A according to a modification of the first embodiment.

Specifically, the second light source 24A according to the modification is provided with, in addition to a light source, an optical system, which are not shown, and the air pump 43 set forth above, an adapter (backside adapter) 24a coupled to one end of a channel C10 whose other end is coupled to the second adapter 21B. The adapter 24a is located to the backside of the universal cord connection side of the second light source 24A. The second light source 24A is provided with a channel C11 communicably joined to the adapter 24a, and a check valve 44a mounted on the channel C1. The check valve 44a is operative to prevent backflow of gas to the adapter 24a.

The second light source 24A is provided with a channel C14 one end of which is coupled to the air pump 43, a check valve 44b mounted on the channel C12, and a channel C13 coupled to a connecting point at which the other end of the channel C11 and that of the channel C12 are joined to each other. The check valve C44b is operative to prevent the carbon dioxide gas supplied from the channel C11 from flowing out to the air pump 43.

The second light source 24A is provided with a front adapter 24b attached to the universal cord connection side of the second light source 24A. The front adapter 24b is located at a position opposite to the backside adapter 24a and coupled to the delivery channel C13 so that the connector 17A of the universal cord 17 is coupled to the front adapter 24b.

Specifically, in the modification of the first embodiment shown in FIG. 7, the second CO₂ delivery channel DC2 for the lumen BC includes the second electromagnetic valve 36, the seventh delivery channel C7, the second flow rate sensor 39, the eighth delivery channel C8, and the second adapter 21B. The second CO₂ delivery channel DC2 also includes the channel C10, the backside adapter 24a, the channel C1, the check valve 44a, the channel C 13, the front adapter 24b, the connector 17A, the universal cord 17 and the flexiblescope 12.

With the modification of the first embodiment shown in FIG. 7, it is possible to securely insufflate the carbon dioxide gas supplied from the gas supply apparatus 21 into the lumen BC through the second light source 24A.

### Second Embodiment

In the first embodiment of the present invention, the gas supply apparatus 21 provides the bifurcating paths of the first CO₂ supply path DC1 for the abdominal cavity AC and the second CO₂ supply path DC2 for the lumen BC through the third and fourth delivery channels C3 and C4.

In contrast, a gas supply apparatus 45 of a second embodiment of the present invention is provided with a switching valve disposed at just upstream of each of the first and second adapters 21A and 21B. The configuration of the gas supply apparatus 45 allows providing a common CO₂ supply path for both the abdominal cavity AC and the lumen BC at the upstream of the switching valve.

In addition, the remaining structures except for the structures related to the switching valve and the common CO₂ supply path are substantially identical to those of the first embodiment described above. Thus, the same reference characters of the gas supply apparatus 21 are assigned to the remaining structures of the second embodiment, and therefore, descriptions thereabout are omitted or simplified.

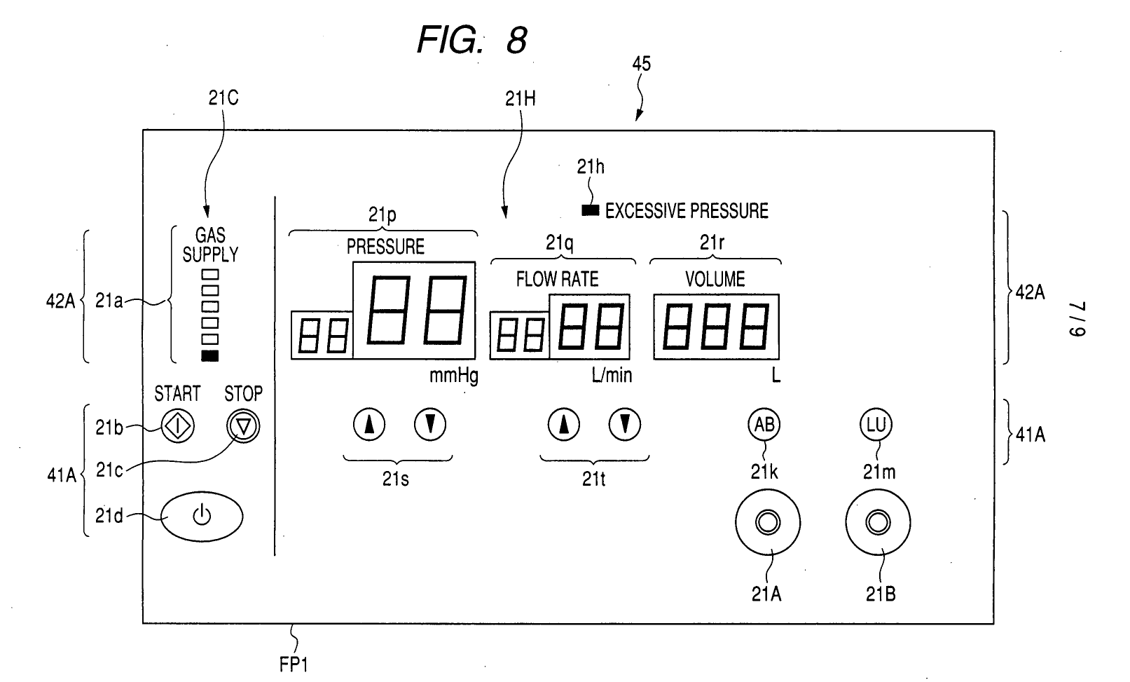

As shown in FIG. 8, a manually operable setting section 41A and a display section 42A are graphically displayed on a front panel FP1 of the gas supply apparatus 45 just like the structure of the first embodiment. The manually operable setting section 41A and display section 42A are divided in, for instance, two graphical setting and display sections 21C and 21H.

The setting and display section 21C serves as a supply source setting and display section that allows the operator to enter instructions related to the carbon dioxide gas supplied from the CO₂ bottle 29. In addition, the setting and display section 21C is designed to display the state of carbon dioxide gas supplied from the CO₂ bottle 29.

The setting and display section 21H serves as a setting and display section for abdominal cavities and lumens.

Specifically, the setting and display section 21H allows the operator to set parameters related to the pressure inside the abdominal cavity AC and the insufflation of carbon dioxide gas thereinto. The setting and display section 21H permits the operator to set parameters related to the pressure inside the lumen BC and the insufflation of carbon dioxide gas thereinto. The setting and display section 21H allows the operator to enter instructions related to the pressure inside the abdominal cavity AC and the insufflation of carbon dioxide gas thereinto. The setting and display section 21H allows the operator to enter instructions related to the pressure inside the lumen BC and the insufflation of carbon dioxide gas thereinto. Furthermore, the setting and display section 21H is designed to display the state of the abdominal cavity AC depending on the carbon dioxide gas insufflated thereinto and that of the lumen BC depending on the carbon dioxide gas insufflated thereinto.

The first and second adapters 21A and 21B are attached to the lower side of the setting and display section 21H of the front panel FP1.

The setting and display section 21C, which is similar to the first embodiment, is provided with the gas remaining volume indicators 21a as the display section 42A. The setting and display section 21C is provided with the gas-supply start button 21b, the gas-supply stop button 21c, and the power switch 21d as the manually operable setting section 41A.

The setting and display section 21H is provided with pressure displays 21p, flow-rate displays 21q, a total volume display 21r, and the excessive pressure indicator 21h as the display section 42A.

The setting and display section 21H is provided with pressure setting buttons 21s, flow-rate setting buttons 21t, the abdominal cavity select button 21k, and the lumen select button 21m.

The pressure setting buttons 21s serve as buttons that allow the operator to send instructions to change the corresponding parameter (the pressure inside the abdominal cavity AC or the lumen BC) to a pressure setting. The flow-rate setting buttons 21t serve as buttons that enable the operator to send instructions to change the corresponding parameter (the flow-rate of the carbon dioxide gas being delivered into the abdominal cavity AC or the lumen BC) to a flow-rate setting.

Specifically, the pressure setting buttons 21s include an up button and a down button. Every time the operator clicks the up button, the pressure setting turns up; every time the operator clicks the down button, the pressure setting turns down. The pressure setting variably set by the up and down buttons 21s is sent to a controller 40A every time at least one of the up and down buttons 21s is operated.

Similarly, the flow-rate setting buttons 21t include an up button and a down button. The flow-rate setting of the carbon dioxide gas being insufflated into the abdominal cavity AC or the lumen BC turns up every time the operator clicks the up button; the flow-rate setting turns down every time the operator clicks the down button. The flow-rate setting variably set by the up and down buttons 21t is sent to the controller 40A every time at least one of the up and down buttons 21t is operated.

The pressure displays 21p include right and left displays arranged facing toward the front panel FP. The right-side display is configured to display a pressure value (in mmHg) based on a measured value of a pressure sensor 37A. The left-side display is configured to display the pressure setting determined based on the operations of, for example, the pressure setting buttons 21s.

The flow-rate displays 21q include right and left displays arranged facing toward the front panel FP. The right-side display is configured to display a flow-rate (in L/min) based on a measured value of a flow-rate sensor 38A. The left-side display is configured to display the flow-rate setting determined based on the operations of, for example, the flow-rate setting buttons 21j.

The total volume display 21r is configured to display a total amount of carbon dioxide gas calculated by the controller 40A based on the measured value of the flow-rate sensor 38A.

The excessive pressure indicator 21h consists of, for example, red LED (light emitting device). The excessive pressure indicator 21h is configured to turn on or flash on and off based on a control signal sent from the controller 40A at anytime the pressure measured by the pressure sensor 37A exceeds a threshold value of the pressure inside the abdominal cavity AC or the lumen BC by a predetermined pressure. The turning-on or the flashing of the excessive pressure indicator 21h allows the operator to visually recognize that the pressure inside the abdominal cavity AC or the lumen BC exceeds the threshold value by a predetermined pressure or more.

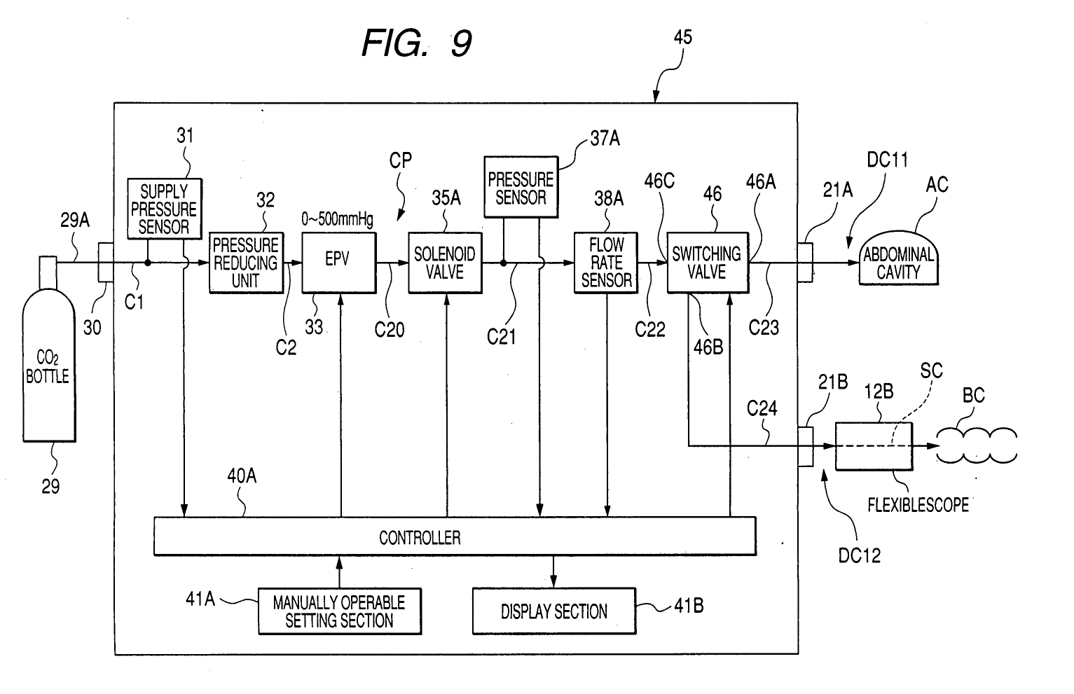

Next, a structure of the gas supply apparatus 45 will be described hereinafter with reference to FIG. 9.

As shown in FIG. 9, the gas supply apparatus 45 of the second embodiment is provided with a common CO₂ supply path CP for both the abdominal cavity AC and the lumen BC, which is coupled to the outlet of the electropneumatic proportional valve 33.

Because the gas supply apparatus 45 of the second embodiment whose elements located at the upstream of the electropneumatic proportional valve 33 are substantially identical to those of the gas supply apparatus 21 of the first embodiment, so that the descriptions of which are omitted or simplified.

In the second embodiment, the common CO₂ supply path CP includes a common delivery channel 20, a solenoid valve 35A, a common delivery channel C21, the flow-rate sensor 38A, and a common delivery channel C22.

The solenoid valve 35A is connected to the outlet of the electropneumatic proportional valve 33 through the common delivery channel C20. The outlet of the solenoid valve 35A is connected to the inlet of the flow-rate sensor 38A through the common delivery channel C21. The pressure sensor 37A is attached to the common delivery channel C21 and configured to detect a pressure of carbon dioxide gas passing through the common delivery channel C21.

The outlet of the flow-rate sensor 38A is connected to one end of the common delivery channel C22.

In addition, the gas supply apparatus 45 is provided with the switching valve 46 whose inlet port is connected to the other end of the common delivery channel C22.

The switching valve 46 has two outlet ports 46A and 46B. The outlet ports 46A and 46B of the switching valve 46 are separated for the abdominal cavity AC and the lumen BC, respectively. The abdominal cavity outlet port 46A is connected to the first adapter 21A through an abdominal cavity output channel C23; the lumen outlet port 46B is connected to the second adapter 21B through a lumen output channel C24.

Incidentally, in the second embodiment, a first CO₂ supply path DC11 directing to the abdominal cavity AC includes the abdominal cavity output channel C23, the first adapter 21A, the abdominal cavity tube 10, and the delivery channel provided in the insufflation guide tube 6. This configuration of the first CO₂ supply path DC11 allows the carbon dioxide gas to be introduced into the abdominal cavity AC therethrough.

In addition, in the second embodiment, a second CO₂ supply path DC12 directing to the lumen BC includes the lumen output channel C24, the second adapter 21B, the lumen tube 22, the connector 17A, the universal cord 17 and the gas delivery channel SC of the flexiblescope 12. This configuration of the second CO₂ supply path DC12 permits the carbon dioxide gas to be introduced into the lumen BC therethrough.

Furthermore, in the second embodiment, a first delivery member of the present invention corresponds to at least the output channel C23 in the first CO₂ supply path DC11. Specifically, the concept of the first delivery member of the present invention can expand to cover the whole of the first CO₂ supply path DC1 depending on aspects of the gas supply apparatus 45.

Likewise, in the second embodiment, a second delivery member of the present invention corresponds to at least the output channel C24 in the second CO₂ supply path DC12. Specifically, the concept of the second delivery member of the present invention can expand to cover the whole of the second CO₂ supply path DC12 depending on aspects of the gas supply apparatus 45.

The supply pressure sensor 31 is electrically connected to the controller 40A. The supply pressure sensor 31 has a function of detecting the pressure of the carbon dioxide gas flowing from the CO₂ bottle 29 to the first delivery channel C1 to send the detected result (detected pressure value) to the controller 40A.

The pressure reducing unit 32 is configured to reduce in pressure the carbon dioxide gas supplied from the CO₂ bottle 29 through the first delivery channel C1.

The electropneumatic proportional valve 33 is electrically connected to the controller 40A. The electropneumatic proportional valve 33 is designed to change its opening in proportional to a voltage or a current as the control signal applied from the controller 40A so as to regulate the pressure and the flow-rate of the carbon dioxide gas flowing therethrough within the corresponding appropriate ranges, respectively.

For example, the electropneumatic proportional valve 33 allows the pressure of the carbon dioxide gas to be regulated within a range from 0 to 500 mmHg based on the control signal applied from the controller 40A.

The solenoid valve 35A is electrically connected to the controller 40A and is operative to open and close based on the control signal sent from the controller 40A.

The pressure sensor 37A is electrically connected to the controller 40A. The pressure sensor 37A has a function of measuring a pressure in the common delivery channel C21 when the electromagnetic valve 35A is closed, thereby sending the measured result to the controller 40A.

The flow rate sensor 38A is electrically connected to the controller 40A. The flow rate sensor 38A has a function of detecting the flow rate of the carbon dioxide gas flowing through the common delivery channel C21. The flow rate sensor 38A is configured to send the detected result to the controller 40A.

The switching valve 46 is electrically connected to the controller 40A. The switching valve 46 has a function of selectively outputting the carbon dioxide gas supplied through the inlet port 46C to either the outlet port 46A or the outlet port 46B.

The controller 40A shown in FIG. 9 is operative to receive the measured values outputted from the supply pressure sensor 31, the pressure sensor 37A, the flow rate sensors 38A. The controller 40A is programmed to execute opening control (pressure control) of the electropneumatic proportional valve 33, opening and closing controls of the solenoid valve 35A, opening and closing controls of the switching valve 46, and display control of the display section 42 based on the received measured values. In addition, the manually operable setting section 41A is electrically connected to the controller 40A. The controller 40A is also programmed to execute opening control (pressure control) of the electropneumatic proportional valve 33, opening and closing controls of the solenoid valve 35A, and display control of the display section 42 based on the instructions sent from the manually operable setting section 41A.

Incidentally, in the second embodiment, the range of the pressure of the carbon dioxide gas to be insufflated into the abdominal cavity AC is preferably 0 to 80 mmHg or thereabout; the range of the flow-rate thereof to be insufflated thereinto is preferably 0.1 to 35 L/min or thereabout. Moreover, in the second embodiment, the range of the pressure of the carbon dioxide gas to be insufflated into the lumen BC is preferably 100 to 500 mmHg or thereabout; the range of the flow-rate thereof to be insufflated thereinto is preferably 1 to 3 L/min or thereabout.

Furthermore, in the second embodiment, as well as the first embodiment shown in FIG. 5, a relief valve can be provided at the midstream of the common delivery channel C22 between the flow rate sensor 38 and the switching valve 46.

Next, operations of the gas supply apparatus 45 of the second embodiment will be described hereinafter.

The gas supply apparatus 45 of the second embodiment is used for the endoscopic surgical system 1 in a similar way as the first embodiment (see FIG. 1).

For example, when carrying out laparoscopic surgery employing the endoscopic surgical system 1, the operator inserts the rigidscope 5 into the inside of the abdominal cavity AC with the flexiblescope 12 being inserted into the lumen BC, such as a large intestine present in the abdominal cavity AC. The operator specifies and treats at least one site to be treated in the abdominal cavity AC and/or the lumen BC based on the first and second images obtained based on the rigidscope 5 and the flexiblescope 12.

Specifically, the operator operates, for example, the abdominal cavity select button 21k and the gas-supply start button 21b so that the instructions corresponding to the buttons 21k and 21b are sent to the controller 40A via the manually operable setting section 41A. The controller 40A executes abdominal-cavity pressure control similar to the first embodiment so that the carbon dioxide gas is supplied into the abdominal cavity AC via the first CO₂ supply path DC1 with its pressure regulated to be suitable for the insufflation inside the abdominal cavity AC.

On the other hand, when the operator operates, for example, the lumen select button 21m and the gas-supply start button 21b so that the instructions corresponding to the buttons 21m and 21b are sent to the controller 40A via the manually operable setting section 41A.

The controller 40A executes lumen flow-rate control similar to the first embodiment based on the instructions so that the carbon dioxide gas is supplied into the lumen BC via the second CO₂ supply path DC2 with its flow-rate regulated to be suitable for the insufflation inside the lumen BC.

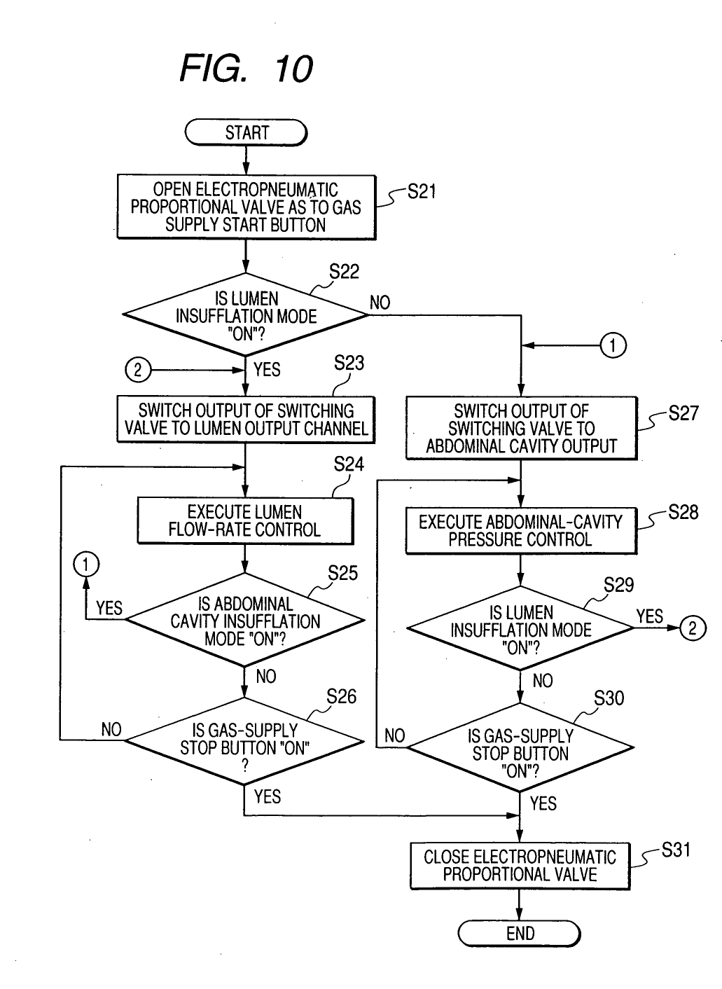

Next, specific control operations of the controller 40A of the gas supply apparatus 45 will be described hereinafter with reference to a flowchart shown in FIG. 10.

In a similar manner to the first embodiment, before laparoscopic surgery, open of the cock of the CO₂ bottle 29 causes the carbon dioxide gas to flow out of the bottle 29 through the high-pressure gas tube 29A. The carbon dioxide gas flows into the gas supply apparatus 45 to be introduced through the first delivery channel C1 to the pressure reducing unit 32.

The carbon dioxide gas is reduced in pressure by the pressure reducing unit 32 to have the predetermined pressure, thereby being guided via the second delivery channel C2 to the inlet of the electropneumatic proportional valve 33.

When actually starting laparoscopic surgery and picking up an image inside the lumen BC with the flexiblescope 12, the operator turns on the gas-supply start button 21b and the lumen select button 21m on the front panel FP 1 of the gas supply apparatus 21. The manually operable setting section 41 provides the instructions corresponding to the turning-on operations of the buttons 21b and 21m to the controller 40A.

The controller 40A receives the instructions corresponding to the turning-on operations of the button 21m and 21b provided from the manually operable setting section 41A to open the electropneumatic proportional valve 33 (FIG. 10; step S21). Incidentally, it is assumed that the electromagnetic valves 35A is kept opened under its initial condition.

Next, the controller 40A determines whether the lumen select button 21m is turned on (step S22).

When the controller 40A determines that the lumen select button 21m is turned on, in other words, the determination in step S22 is YES, the controller 40A enters the lumen insufflation mode. In the lumen insufflation mode, the controller 40A sends the control signal to the switching valve 46 so that the switching valve 46 switches its output to the lumen output port 46B (step S23). After the switching operation of the switching valve 46, the controller 40A sends the control signal based on the lumen insufflation mode to execute the lumen flow-rate control, which is similar to the first embodiment (step S24).

As described in the first embodiment, during the lumen flow-rate control, the carbon dioxide gas whose pressure is reduced to the predetermined pressure by the pressure reducing unit 32 is regulated so that the pressure and the flow-rate of the carbon dioxide gas are set within the corresponding predetermined ranges suitable for the insufflation of the lumen BC, respectively.

The carbon dioxide gas with its pressure and flow-rate being regulated each is introduced through the common delivery channel C20, the solenoid valve 35A, common delivery channel C21, the flow-rate sensor 38A, and the common delivery channel C22 to the switching valve 46.

Because the output of the switching valve 46 is switched to the lumen output port 46B, the carbon dioxide gas does not flow into the abdominal cavity output channel C23 but flows into the lumen output channel C24. After that, the carbon dioxide gas is delivered through the lumen output channel C24, the second adapter 21B, the lumen tube 22, the connector 17A, the universal cord 17 and the gas delivery channel SC provided in the flexiblescope 12 to the lumen BC.

While the carbon dioxide gas is delivered through the second CO₂ supply path DC12 toward the lumen BC, close of the through hole of the manipulator 13 by the operator allows the carbon dioxide gas to be supplied into the lumen BC.

While the carbon dioxide gas is supplied into the lumen BC through the second CO₂ supply channel DC12, the flow-rate sensor 38A measures the flow rate of the carbon dioxide gas flowing across the solenoid valve 35A through the common delivery channel C21. The flow-rate sensor 38A sends the measured result to the controller 40A. The controller 40A receives the measured result. The controller 40A controls the opening of the electropneumatic proportional valve 33 based on the measured result. The control of the opening of the valve 33 causes the flow-rate of the carbon dioxide gas into the lumen BC to be regulated within the predetermined range of, for example, approximately 1 to 3 L/min set forth above, thereby controlling the flow rate in the lumen BC and the pressure inside it.

During the lumen flow-rate feedback control, the controller 40A determines whether the abdominal cavity select button 21k is turned on (step S25). When it is determined that the abdominal cavity select button 21k is not turned on, in other words, the determination in step S25 is NO, the controller 40A determines whether the gas-supply stop button 21c is turned on (step S26).

When the operator decides to continuously execute the lumen flow-rate control, because no switches 21k and 21b are manipulated, each of the determinations in steps S25 and S26 is NO so that the controller 40A continues executing the lumen flow-rate control.

On the other hand, for example, when the operator wants to shift the operation mode from the lumen insufflation mode to the abdominal-cavity insufflation mode, the operator turns on the abdominal cavity select button 21k on the front panel FP1 of the gas supply apparatus 45. The manually operable setting section 41A provides the instruction corresponding to the turning-on operation of the button 21k to the controller 40A.

In this case, the controller 40A receives the instruction corresponding to the turning-on of the button 21k provided from the manually operable setting section 41A so as to determine that the abdominal cavity select button 21k is turned on (the determination in step S25 is YES), automatically shifting into the abdominal-cavity insufflation mode.

Specifically, the controller 40A sends the control signal to the switching valve 46 so that the switching valve 46 switches its output to the abdominal cavity output port 46A (step S27).

After the switching operation, the controller 40A sends the control signal based on the abdominal-cavity insufflation mode to the electropneumatic proportional valve 33 to execute the abdominal-cavity pressure control, which is similar to the first embodiment (step S28).

As described in the first embodiment, during the abdominal-cavity pressure control, the carbon dioxide gas whose pressure is reduced to the predetermined pressure by the pressure reducing unit 32 is regulated so that the pressure and the flow-rate of the carbon dioxide gas are set within the corresponding predetermined ranges suitable for the insufflation of the abdominal cavity AC, respectively.

The carbon dioxide gas with its pressure and flow-rate being regulated each is introduced through the common delivery channel C20, the solenoid valve 35a, the common delivery channel C21, the flow-rate sensor 38A, and the common delivery channel C22 to the switching valve 46.

Because the output of the switching valve 46 is switched to the abdominal cavity output port 46A, the carbon dioxide gas does not flow into the lumen output channel C24 but flows into the abdominal cavity output channel C23. After that, the carbon dioxide gas is delivered into the abdominal cavity AC through the abdominal cavity output channel C23, the first adapter 21A, the abdominal cavity tube 10 and the delivery channel provided in the abdominal-cavity guide tube 6.

While the carbon dioxide gas is delivered through the first CO₂ supply path DC11 into the abdominal cavity AC, the flow rate sensor 38A measures the flow rate of the carbon dioxide gas flowing across the solenoid valve 35A through the common delivery channel C21. The flow-rate sensor 38A sends the measured result to the controller 40A.

Similarly, while the carbon dioxide gas is delivered through the first CO₂ supply path DC1 into the abdominal cavity AC, the pressure sensor 37A measures the pressure inside the abdominal cavity AC when the electromagnetic valve 35A is closed. The pressure sensor 37A sends the measured result to the controller 40A.

The controller 40A receives the measured results provided from the sensors 37A and 38a. The controller 40A controls the opening of the electropneumatic proportional valve 33 based on the measured results to regulate the pressure of the carbon dioxide gas and the flow-rate thereof supplied into the abdominal cavity AC within the corresponding predetermined range of approximately 0 to 80 mmHg and that of approximately 0.1 to 35 L/min, respectively.

During the abdominal-cavity pressure feedback control, the controller 40A determines whether the lumen select button 21m is turned on (step S29). When it is determined that the lumen select button 21m is not turned on, in other words, the determination in step S29 is NO, the controller 40A determines whether the gas-supply stop button 21c is turned on (step S30).

When the operator decides to continuously execute the abdominal-cavity pressure control, because no switches 21m and 21b are manipulated, each of the determinations in steps S29 and S30 is NO so that the controller 40A continues executing the abdominal-cavity pressure control shown in step S28.

On the other hand, for example, when the operator wants to shift the operation mode from the abdominal-cavity insufflation mode to the lumen insufflation mode, the operator turns on the lumen select button 21m on the front panel FP1 of the gas supply apparatus 45 again. The manually operable setting section 41A provides the instruction corresponding to the turning-on operation of the button 21m to the controller 40A again.

The controller 40A receives the instruction corresponding to the turning-on of the button 21m provided from the manually operable setting section 41A so as to determine that the lumen select button 21m is turned on (the determination in step S29 is YES), automatically shifting into the lumen insufflation mode and executing the operations shown in step S23 and thereafter.

Thus, the body-cavity pressure control in the abdominal cavity insufflation mode and the lumen flow-rate control in the lumen insufflation mode are carried out by the controller 40A. During the body-cavity pressure control and the lumen flow-rate control, when the operator determines that it eliminates the need for supplying the carbon dioxide gas into both the abdominal cavity AC and the lumen BC, the operator turns on the gas-supply stop button 21c on the front panel FP1. For example, when observations and treatments for at least one site to be treated are completed, the operator turns on the gas-supply stop button 21c on the front panel FP1. The manually operable setting section 41A provides the instruction corresponding to the turning-on operation of the button 21c to the controller 40A.

Accordingly, each of the determinations of the controller 40A in steps S26 and S30 is YES, so that the controller 40A sends the control signal to the electropneumatic proportional valve 33 to close it (step S31), thereby terminating the operations shown in FIG. 10.

As described above, in the second embodiment, the controller 40A executes the abdominal-cavity pressure control in the abdominal-cavity insufflation mode and the lumen flow-rate control in the lumen insufflation mode depending on the operator's needs. This allows the carbon dioxide gas whose pressure and flow-rate are regulated suitably to the abdominal cavity AC to be supplied thereinto, as well as the carbon dioxide gas whose pressure and flow-rate are regulated suitably to the lumen BC to be supplied thereinto.

The operator, therefore, can easily rapidly specify at least one site to be treated in the patient 3 while observing the first image corresponding to the inside of the abdominal cavity AC being sufficiently insufflated and the second image corresponding to the inside of the lumen BC being sufficiently insufflated. It is possible for the operator to treat the specified at least one site in the patient using, for example, the electrical scalpel probe 7.

Accordingly, as well as the first embodiment, the second embodiment of the present invention makes the structure of the gas supply apparatus 45 compact, and allows the cost thereof to decrease. In addition, the second embodiment of the present invention enables preparations for setting up the gas supply apparatus 45 to be reduced, and makes it possible to reduce a space where the apparatus 45 is occupied in an operating room, in other words, to increase empty space in the operating room.

Especially, in the second embodiment, providing the switching valve 46 to closely upstream of the first and second adapters 21A and 21B allows commonality of an upstream CO₂ supply path of the switching valve 46 between the abdominal cavity AC and the lumen BC, as the common CO₂ supply path CP. The structure makes it possible to reduce the number of elements of the gas supply apparatus 45 as compared with those of the apparatus 21 according to the first embodiment. As a result, it is possible to offer simplified manufacturing of the gas supply apparatus 45 and to reduce the manufacturing cost thereof.

Incidentally, the structure of the second embodiment needs not necessarily the electromagnetic valve 35.

Moreover, in the structure of the second embodiment, the switching valve 46 is disposed closely upstream the first and second adapters 21A and 21B, but the present invention is not limited to the structure. Specifically, the switching valve 46 can be disposed in the midstream of a delivery channel between the downstream side of electropneumatic proportional valve 33 and each of the first and second adapters 21A and 21B. In this modification, a CO₂ supply path extending from the downstream of the switching valve 46 branches into a first CO₂ supply path and a second CO₂ supply path so that a flow-rate sensor may be required for each branch depending on needs.

Moreover, even in the structure of the second embodiment, the second light source 24A, shown in FIG. 7, may be employed as a part of the second CO₂ supply path DC12 for the lumen BC.

In addition, in each of the first and second embodiments and their modifications, the controller 40 (40A) determines that its operating mode is in the abdominal-cavity insufflation mode while the lumen select button is in off state; its operating mode is in the lumen insufflation mode while the abdominal cavity select button is in off state. The present invention, however, is not limited to the configuration. Specifically, while one of the lumen select button and the abdominal cavity select button is in off state, the controller 40 (40A) can stand by until the other of the buttons is turned on.

Moreover, in each of the first and second embodiments and their modifications, the controller 40 or 40A carries out the gas-supply control operations shown in FIG. 6 or FIG. 10, but the system controller 25 can execute them.

In addition, in each of the first and second embodiments and their modifications, the rigidscope and the flexiblescope are used as observation devices for observing the inside of a specimen, but the present invention is not limited to the structure. Specifically, other types of endoscopes, such as a wireless capsule endoscope or the like, or other observation devices except for endoscopes, each of which is configured to be inserted into the inside of a specimen, can be used for observing the inside of the specimen.

Furthermore, it should be noted that the term “body cavity” means not only a cavity that originally exists in the body of a specimen, but also a cavity (space) to be artificially formed in the body of a specimen with medical instruments.

For example, the term “body cavity” according to the specification includes, as the former means, an abdominal cavity, a lumen, and the like. In the specification, the lumen includes upper alimentary tracts (esophagus, stomach, or the like), lower alimentary tracts (large intestine, small intestine, or the like), a bladder, and a uterus.

In addition, the term “body cavity” according to the specification includes, as the later means, a cavity to secure the field of an endoscope during surgery, such as subcutaneous cavity and the like.

While there has been described what is at present considered to be these embodiment and modifications of the invention, it will be understood that various modifications which are not described yet may be made therein, and it is intended to cover in the appended claims all such modifications as fall within the true spirit and scope of the invention.

1. A gas supply apparatus comprising:
a supplier supplying predetermined gas;
a first delivery member delivering the predetermined gas to a first body cavity inside a specimen;
a second delivery member delivering the predetermined gas to a second body cavity inside the specimen;
a pressure regulator coupled to the supplier to receive the predetermined gas supplied from the supplier, the pressure regulator regulating a pressure of the received predetermined gas to a first pressure and a second pressure, the first pressure being suitable for the first body cavity, the second pressure being suitable for the second body cavity;
a switching unit coupled to the pressure regulator, and the first and second delivery members, and configured to switch output of the predetermined gas, whose pressure is regulated by the pressure regulator, to any one of the first and second delivery members; and
a controller electrically connected to the pressure regulator and the switching unit, and operative to control the pressure regulator and the switching unit so that the predetermined gas with the first pressure is supplied to the first delivery member and the predetermined gas with the second pressure is supplied to the second delivery member.

2. A gas supply apparatus according to claim 1, wherein the switching unit comprises:
a first opening and closing valve connected to the pressure regulator and the first delivery member, and electrically connected to the controller; and
a second opening and closing valve connected to the pressure regulator and the second delivery member and electrically connected to the controller.

3. A gas supply apparatus according to claim 2, wherein the controller is operative to:
close the first opening and closing valve to allow the pressure regulator to regulate the pressure of the predetermined gas to the second pressure; and
close the second opening and closing valve to allow the pressure regulator to regulate the pressure of the predetermined gas to the first pressure.

4. A gas supply apparatus according to claim 1, wherein the pressure regulator comprises:
a pressure reducing unit configured to reduce the pressure of the predetermined gas supplied from the supplier; and
an electropneumatic regulator coupled to the pressure reducing unit and electrically connected to the controller, the electropneumatic regulator regulating the reduced pressure of the predetermined gas to any one of the first and second pressures.

5. A gas supply apparatus according to claim 4, further comprising a pressure sensor connected to the first delivery member and electrically connected to the controller, the pressure sensor measuring a pressure inside the first body cavity,
wherein the first pressure takes any value within a first predetermined pressure range, and
while closing the second opening and closing valve, the controller controls the electropneumatic regulator depending on the measured pressure inside of the first body cavity to keep the pressure of the predetermined gas delivered through the first delivery member within the first predetermined pressure range.

6. A gas supply apparatus according to claim 4, further comprising a flow-rate sensor electrically connected to the controller and configured to measure a flow-rate of the predetermined gas flowing through the second delivery member, wherein, while closing the first opening and closing valve, the controller controls the electropneumatic regulator depending on the flow-rate measured by the flow-rate sensor to keep a flow-rate of the predetermined gas delivered through the second delivery member within a predetermined flow-rate range.

7. A gas supply apparatus according to claim 1, wherein the first body cavity is an abdominal cavity inside the specimen, and the second body cavity is a lumen inside the specimen.

8. A gas supply apparatus according to claim 1, wherein the switching unit comprises a switching valve coupled to the pressure regulator and the first and second delivery members, and electrically connected to the controller, the switching valve switching the output of the predetermined gas, whose pressure is regulated by the pressure regulator, to any one of the first and second delivery members.

9. A gas supply apparatus according to claim 8, wherein the controller controls the pressure regulator to regulate the pressure of the predetermined gas to the first pressure, thereby causing the switching valve to supply the predetermined gas with the first pressure to the first delivery member, and controls the pressure regulator to regulate the pressure of the predetermined gas to the second pressure, thereby causing the switching valve to supply the predetermined gas with the second pressure to the second delivery member.

10. A gas supply apparatus according to claim 8, wherein the switching unit further comprises a common deliver member coupled to the pressure regulator, wherein the switching valve is coupled to the common delivery member and the first and second delivery members, respectively, so that the predetermined gas, whose pressure is regulated by the pressure regulator, is delivered through the common delivery member, the switching valve switching the output of the predetermined gas delivered through the common delivery member any one of the first and second delivery members.

11. A gas insufflating apparatus for insufflating predetermined gas supplied from a supplier to a first body cavity of a specimen through a first delivery member and to a second body cavity of the specimen through a second delivery member, the gas insufflating apparatus comprising:
means for switching output of the predetermined gas to any one of the first and second delivery members;
means for regulating a pressure of the predetermined gas to a first pressure suitable for the first body cavity when output of the predetermined gas is switched to the first delivery member by the switching means; and
means for regulating the pressure of the predetermined gas to a second pressure suitable for the second body cavity when the output of the predetermined gas is switched to the second delivery member by the switching means.

12. An observation system comprising:
a gas supply apparatus comprising:
a supplier supplying predetermined gas;
a first delivery member delivering the predetermined gas to a first body cavity inside a specimen;
a second delivery member delivering the predetermined gas to a second body cavity inside the specimen;
a pressure regulator coupled to the supplier to receive the predetermined gas supplied from the supplier, the pressure regulator regulating a pressure of the received predetermined gas to a first pressure and a second pressure, the first pressure being suitable for the first body cavity, the second pressure being suitable for the second body cavity;
a switching unit coupled to the pressure regulator, and the first and second delivery members, and configured to switch output of the predetermined gas, whose pressure is regulated by the pressure regulator, to any one of the first and second delivery members; and
a controller electrically connected to the pressure regulator and the switching unit, and operative to control the pressure regulator and the switching unit so that the predetermined gas with the first pressure is supplied to the first delivery member and the predetermined gas with the second pressure is supplied to the second delivery member; and
an observation device integrated with a gas delivery channel and configured to be inserted into the second body cavity of the specimen to observe an inside of the second body cavity, the gas delivery channel serving as part of the second delivery member.

13. A method of supplying gas using a first delivery member connected into an inside of a first body cavity of a specimen and a second delivery member connected into an inside of a second body cavity of the specimen, the method comprising:
switching output of the predetermined gas to any one of the first and second delivery members;
regulating a pressure of the predetermined gas to a first pressure suitable for the first body cavity when the output of predetermined gas is switched to the first delivery member by the switching; and
regulating the pressure of the predetermined gas to a second pressure suitable for the second body cavity when the output predetermined gas is switched to the second delivery member by the switching.
